SEQUENCE LISTING (145 sequences)

SEQ ID NO: 1 - DNA, 22 bp, Glycine max
gggttgagttgcagaggctgaa

SEQ ID NO: 2 - DNA, 22 bp, Glycine max
atgaccatgaatgggtccaagc

SEQ ID NO: 3 - DNA, 15 bp, Glycine max
cttcacctaaatcag

SEQ ID NO: 4 - DNA, 15 bp, Glycine max
cttcagctaaatcag

SEQ ID NO: 5 - DNA, 538 bp, Glycine max
tycybbkddwwmhrggcywtgacywtkwatkkgtycaagctcgaagaaagcttcasctaaatcaggtgtgacaaakcacatgaagggcttcagcctctgcaactcaaccctaaaagtaaatgggcaagtcatcctctcccaagtccccaagaacgtaaccctcaccccatgcacctacgacactcacaccaccggatgcttcctcggtttccacgccacctccccaaaatcccgccacgtggcacccttaggacagcttaaaaacataagcttcacttccatcttccggttcaaggtttggtggaccactctctggaccggctccaacggccgcgacctggaaaccgaaacccaattcctcatgctccaatcccacccttatgttctcttcctacccatcctccaacccccatttcgcgcctcgctgcagcctcactcagacgacaacgttgcggtgtgtgtggagagcggctccadccrsgtaacagcctcatcattcgacactgtcgtctacttgcacgcaggggacaacccwksc

SEQ ID NO: 6 - DNA, 26 bp, Glycine max
ccaattaaaattttcctcagatccta

SEQ ID NO: 7 - DNA, 22 bp, Glycine max
tggtttgatgggtgcttaattt

SEQ ID NO: 8 - DNA, 15 bp, Glycine max
cagcatctcaggcca

SEQ ID NO: 9 - DNA, 14 bp, Glycine max
cagcatcacaggcc

SEQ ID NO: 10 - DNA, 401 bp, Glycine mas
ttcacatcaaaattttgtactttcctaattctgctggtccgtactgacggatttttaccgttttaaataactgtaatggtggtgtgtgtaagaatgggacagtcagtggccaaaaatccaagaatcaattatccacccaaccaccaattaaaattttcctcagatcctagggtcccatgattccaaccaaaagcagcatcwcaggccaaaaaattaagcacccatcaaaccactttttagcttttccaacttatgttcctcttcccccctgcaaaaataatgtctagaccactgcaggctctcggagctttttctgaagattatgtgccacagcatcaataaattcctccgtgttcaagtaaaattcccttgataccctgcacatcatgcatgctcgggtg

SEQ ID NO: 11 - DNA, 25 bp, Glycine max
tcgatctcaaaatgctaaatcttgt

SEQ ID NO: 12 - DNA, 26 bp, Glycine max
ttgtgcattgaaagttttgacttctt

SEQ ID NO: 13 - DNA, 18 bp, Glycine max
tctcattcaagtcccctg

SEQ ID NO: 14 - DNA, 14 bp, Glycine max
tcattcaggtcccc

SEQ ID NO: 15 - DNA, 401 bp, Glycine max
ccgaaacctcccaagtaccagtaattttaatacgtagtgctgcagaatgtgatgaagaggagaaatcatccagcttgaatctgtcttcaaaaaatcctcaagtagataatggggtgcaattggatctcaagtcaaaatctcgatctcaaaatgctaaatcttgtagatctcgagatgttgatgctccatcttctcattcargtcccctgccttatacaaatgttaagaagtcaaaactttcaatgcacaaagagtcaaaatctgatcttcaaaggccaaaaggggatgagcagggacctaaagataaggtaactgcakaagatctgaaactcggaagcgaagtaactgccaaagtctcgcaaattggtgcacatgggttagtgttggatttgggtggagga

SEQ ID NO: 16 - DNA, 20 bp, Glycine max
acccgggaatgtgaatgata

SEQ ID NO: 17 - DNA, 19 bp, Glycine max
tatccaatggtggggatgg

SEQ ID NO: 18 - DNA, 16 bp, Glycine max
aagacatcattgctca

SEQ ID NO: 19 - DNA, 16 bp, Glycine max
aagacatgattgctca

SEQ ID NO: 20 - DNA, 484 bp, Glycine max
  misc_feature (1)..(485): n = A, C, G, or T
aatttctgaaactcagtagcaacgttctacatgaattttctatttaaagattcacaaatgtaataagccacttgtcatagcatggtataccaaggcagagtttggtaacactagctaagtagccatgcgwtttacacagatactaacagaataagacataagaacagaatgtgaggaatattttagggaggctctgaatcaatgttagtttgaggaatgcaatttccttattcatgacttattttkggtagggttgtcacattacccgggaatgtgaatgatactgacattgtaaaaagacatsattgctcacacccttaatgatctagcccccccaatgccccatccccaccattggatagccttcactaccactgtcaatgtcactgtcacttccatcactcaataaattgtcattaggttgaaattttgaagaaaaaaaaaatccnattttccctcnttancctatttttggtctcaattt

SEQ ID NO: 21 - DNA, 25 bp, Glycine max
gtttatccagactgctttcttttgt

SEQ ID NO: 22 - DNA, 27 bp, Glycine max
tgctcctagataggaatatttaacacg

SEQ ID NO: 23 - DNA, 15 bp, Glycine max
tctgacagccttctt

SEQ ID NO: 24 - DNA, 18 bp, Glycine max
cagccttcttacttgtta

SEQ ID NO: 25 - DNA, 766 bp, Glycine max
  misc_feature (1)..(766): n = A, C, G, or T
atctaattggctaaaactaaaacactaatctaaggtggctaatggctacaccttgttgctttcatcctaggataggccaactccctagaggcagctccatctaatgggcgaaaacttatgtctcaagggacggcttcatcatgttggtagtctttgtttgcctctcacaggcggcactttgctcctagataggaatatttaacacgacacattctaacaagtaagaaggctgtcagacaacaaaagaaagcagtctggataaacattaaacatattatcttccatttctacaagattacgttaygaaagcaataacttgcctgcaataaacaagatggaatttggatataaccctgtagcaagacgttgactttttcgagagaattatgtggtacaggagtaatgacataaagcacacctttgactgtgtcaattcccctcacaatccctgaaacataagaaatcaagatgtgtttaacaattcatatgcttagaattattgaaatgcataactactcaactagaacagtatctgtatgatttgtttccatcatggatacacttttctttttttatcttttacatttatggaagtatgatattacaaagctaagagacactggtaaacttgttacaaccaatatagacagacatacnagatctaatgaagcwgtgaaatttaaggactacattgttgaagattctgaattaaattacattactgaagtgctgagttaagttctcagcatatctcantatacatata

SEQ ID NO: 26 - DNA, 25 bp, Glycine max
gcattaatagccacctcgcacctaa

SEQ ID NO: 27 - DNA, 44 bp, Glycine max
gaaggtgaccaagttcatgctttgtggatgaaaagcaagagcgc

SEQ ID NO: 28 - DNA, 46 bp, Glycine max
gaaggtcggagtcaacggattctttgtggatgaaaagcaagagcgt

SEQ ID NO: 29 - DNA, 314 bp, Glycine max
tggtgatgaagaagacgacgacggcggtggctcctttgtggatgaaaagcaagagcgytaggggtggtattaggtgcgaggtggctattaatgcggtggatgagtcgacgacgtcaccggagkcgaagattggagcgcgtgtgaaggtgaaggcgggtgtaaaggtgtaccacgtccccaaagtagccgagcttgacctcacgggtctggaaggcgagatcaagcagtatgttggcctctggaacggtaagcgaatctccgccaatcttccttacaaggttcagtttctcaccgacatcccagctcgtggtcct

SEQ ID NO: 30 - DNA, 25 bp, Glycine max
catgcctcctgaatcccaatagctt

SEQ ID NO: 31 - DNA, 48 bp, Glycine max
gaaggtgaccaagttcatgctatgtcaatacttggagtacacatcatc

SEQ ID NO: 32 - DNA, 49 bp, Glycine max
gaaggtcggagtcaacggattcatgtcaatacttggagtacacatcatt

SEQ ID NO: 33 - DNA, 491 bp, Glycine max
  misc_feature: n = A, C, G, or T
ggsctagcttttatctttgaaganactactactaagttgaarttaaaaaaaaattctaatgttgtttggtattttatggttttttaacttaattgcaaacttctacattactagtgattgtcaaacaattggcatgcactacaaacatgtcaatacttggagtacacatcatyaagattagtgagcacaaagctattgggattcaggaggcatggttttgagaatgcatatggagcttgatacccaggaaaacatcacttaactaagagcaagctttgaggatgataggatcgtggccgagactaaraatgagcctcatgaggtaatagaatgaaacaagggcatgcatattgatgaggaaatagtagcagatcacaactttgaaggaatagaaattgggcgagagcaggtggttgaacaagagatcaccaaagaggagctttgtgacaaacatggaggttgtcaatcaggccatgcatggtcatagctkk

SEQ ID NO: 34 - DNA, 30 bp, Glycine max
aaaattctatggaaatggatcccctacatt

SEQ ID NO: 35 - DNA, 46 bp, Glycine max
gaaggtgaccaagttcatgctgagcgattttcatccagcagtttta

SEQ ID NO: 36 - DNA, 46 bp, Glycine max
gaaggtcggagtcaacggattgagcgattttcatccagcagttttg

SEQ ID NO: 37 - DNA, 401 bp, Glycine max
gaatctcctacttgccatgccacaacaagctatagaattttggatagggattccttgcagtcacagattttcagcattcacacattcagctactcaaaatcgttggatgtagatctgacaactcatattttatgccgataaacctaacttaaaaataagcatgcaagatctaaactgagcgattttcatccagcagttttrcacaaatgtatgaatgtagsggatccatttccatagaattttatggttaaccacccaagctgaaaagtggggatttgatagaaacgacaaaggggcctgacccaactcccaatgctgaacccccagatttcacagaacataccttccatttcctctaagcacttgccaatatgcataattttaaaaaatcaagcagcaat

SEQ ID NO: 38 - DNA, 22 bp, Glycine max
gcatgcaagatctaaactgagc

SEQ ID NO: 39 - DNA, 20 bp, Glycine max
tggaaatggatcccctacat

SEQ ID NO: 40 - DNA, 17 bp, Glycine max
ccagcagttttacacaa

SEQ ID NO: 41 - DNA, 16 bp, Glycine max
ccagcagttttgcaca

SEQ ID NO: 42 - DNA, 401 bp, Glycine max
gaatctcctacttgccatgccacaacaagctatagaattttggatagggattccttgcagtcacagattttcagcattcacacattcagctactcaaaatcgttggatgtagatctgacaactcatattttatgccgataaacctaacttaaaaataagcatgcaagatctaaactgagcgattttcatccagcagttttrcacaaatgtatgaatgtaggggatccatttccatagaattttatggttaaccacccaagctgaaaagtggggatttgatagaaacgacaaaggggcctgacccaactcccaatgctgaacccccagatttcacagaacataccttccatttcctctaagcacttgccaatatgcataattttaaaaaatcaagcagcaat

SEQ ID NO: 43 - DNA, 26 bp, Glycine max
aaaataagcatgcaagatctaaactg

SEQ ID NO: 44 - DNA, 20 bp, Glycine max
atggaaatggatcccctaca

SEQ ID NO: 45 - DNA, 15 bp, Glycine max
ccagcagttttacac

SEQ ID NO: 46 - DNA, 15 bp, Glycine max
ccagcagttttgcac

SEQ ID NO: 47 - DNA, 758 bp, Glycine max
  misc_feature (1)..(758): n = A, C, G, or T
tttacacacaaaaataaagaaatttttggaacttgaatctcctacttgccatgccacaacaagctatagaattttggatagggattccttgcagtcacagattttcagcattcacacattcagctactcaaaatcgttggatgtagatctgacaactcatattttatgccgataaacctaacttaaaaataagcatgcaagatctaaactgagcgattttcatccagcagttttrcacaaatgtatgaatgtaggggatccatttccatagaattttatggttaaccacccaagctgaaaagtggggatttgatagaaacgacaaaggggcctgacccaactcccaatgctgaacccccagatttcacagaacataccttccatttcctctaagcacttgccaatatgcataattttaaaaaatcaagcagcaatcaaagrgtttttctattatgcgccagcatcggcaggtgcacgataaaaagtcaataatagaaacaaaaatcattagcaaagagagcatttatcagattgaaaacacaaaagtcactaaatgcttgcatttgttgagtgatttaaaacattcaacatatatatttcatgaacacacgcacacacagtagagaaccccatactgaaaaaaactaaagtgaaatagtgtgtgagtgtggtgtgwgtgtgtgtgtgtatattctcccccagcattgaaagatagcaaacacccccntcgagaggactctcaaaattatggcatggca

SEQ ID NO: 48 - DNA, 21 bp, Glycine max
catcgattattcccacaaacc

SEQ ID NO: 49 - DNA, 27 bp, Glycine max
actagttatgtgatggttgatcttctg

SEQ ID NO: 50 - DNA, 14 bp, Glycine max
ctcacacgctttct

SEQ ID NO: 51 - DNA, 14 bp, Glycine max
tcacacactttctc

SEQ ID NO: 52 - DNA, 401 bp, Glycine max
cctctcgrtggttctcacattatgatacttcactgaacatccttgttggtcaaaaccctycaccagtcaaaattctctgtagatagccctttytgagacctcgacaaccaactctgacagtgctcaactgctttcaagatcatcgattattcccacaaaccaacaagagatttttcaacatgttttatccttactcacacrctttctcggaaacttcccagaagatcaaccatcacataactagtccaagtcacccacaaaccaatatgcttctaagattaatgattgtcccacaaaccaacacaagacttttcaaygtgatttgtcctcactcacgttttttggaaaactttctaaaagrtcacccatctcataactacttcaaaccaaacacgcttaat

SEQ ID NO: 53 - DNA, 26 bp, Glycine max
gggacacgttaatcagggacaaagtt

SEQ ID NO: 54 - DNA, 46 bp, Glycine max
gaaggtgaccaagttcatgctaaacggttcagatcaaaacacgtgc

SEQ ID NO: 55 - DNA, 46 bp, Glycine max
gaaggtcggagtcaacggattaaacggttcagatcaaaacacgtgg

SEQ ID NO: 56 - DNA, 401 bp, Glycine max
aaagaattatttatttattttatatttaaaaaccatttaaacataattttgtaaaataaaattaatgttatattaaccctaattcactttgtagagtatgatttctacatttctttccatttcttttccttactcctcccgaaaacaaaaccagctctagagctggtgttgcaatcaaacggttcagatcaaaacacgtgsactgatttgtgagaaactttgtccctgattaacgtgtccccagcgggagtggaacacagaaattttcacagaaaaaactgcgcgagtatggaaagtttttccatgcttgtgttgttgcattgtccctgaatttgtacttgattctgggacgtcactcagtctgagtctgtttctgcaaatttcaaaaccctctctctcgt

SEQ ID NO: 57 - DNA, 28 bp, Glycine max
gcttgatgatttagattcgaactgttat

SEQ ID NO: 58 - DNA, 21 bp, Glycine max
tcaagacctattttgcgcttt

SEQ ID NO: 59 - DNA, 19 bp, Glycine max
ttgacaatttaactgatcc

SEQ ID NO: 60 - DNA, 18 bp, Glycine max
acaatttaaccgatccta

SEQ ID NO: 61 - DNA, 401 bp, Glycine max
tggaataaaatctcttcgtatacccmtctatagtttaggtaagaccaaaagggtcactataaatattcactatgatcggcttaacggtggtgaattggaatagatgcttgatgatttagattcgaactgttattatacaccaaaaagataaggatcactatgaattctctgtagtctaattgttcttttgacaatttaacygatcctaaaatatgaaaatattagataaaagcgcaaaataggtcttgattcagttctctgttagtaacaacctaaattgcaaacacatctctagtgtgaawagacattgataccttaaaaaacactagtcaacccaaaagcwtgagctttgtaatcattcatgtttgttattgccaatktggaatattgtccttttggca

SEQ ID NO: 62 - DNA, 23 bp, Glycine max
aaggcatgcatagcactttaact

SEQ ID NO: 63 - DNA, 28 bp, Glycine max
ccatcttcaattgtacagtttcatactt

SEQ ID NO: 64 - DNA, 17 bp, Glycine max
aataccctggttgaatc

SEQ ID NO: 65 - DNA, 15 bp, Glycine max
ataccccggttgaat

SEQ ID NO: 66 - DNA, 401 bp, Glycine max
actttctaaaagrtcacccatctcataactacttcaaaccaaacacgcttaattgtgaaattcttaagtgataactaccgaaaagtagatgcatcttattggtataagaaataccaattaattcttttaaaccatcttcaattgtacagtttcatacttgtacaatctctggatccccctcattctgatgtgattcaaccrgggtattatactcaaaaacaacaagaatcaagtataaatagttaaagtgctatgcatgccttttgtttttgccacttcccattgattaaaaaaactcacttgagagtggcttcagcactttccacagtaactctgtcatcagtagcatcacgattttgaagccccaaatcaaagtactttatgttcaaatccaggtaagg

SEQ ID NO: 67 - DNA, 29 bp, Glycine max
tagttatgtgatggttgatcttctgggaa

SEQ ID NO: 68 - DNA, 50 bp, Glycine max
gaaggtgaccaagttcatgcttttcaacatgttttatccttactcacaca

SEQ ID NO: 69 - DNA, 47 bp, Glycine max
gaaggtcggagtcaacggattcaacatgttttatccttactcacacg

SEQ ID NO: 70 - DNA, 401 bp, Glycine max
cctctcggtggttctcacattatgatacttcactgaacatccttgttggtcaaaacccttcaccagtcaaaattctctgtagatagccctttctgagacctcgacaaccaactctgacagtgctcaactgctttcaagatcatcgattattcccacaaaccaacaagagatttttcaacatgttttatccttactcacacrctttctcggaaacttcccagaagatcaaccatcacataactagtccaagtcacccacaaaccaatatgcttctaagattaatgattgtcccacaaaccaacacaagacttttcaacgtgatttgtcctcactcacgttttttggaaaactttctaaaaggtcacccatctcataactacttcaaaccaaacacgcttaat

SEQ ID NO: 71 - DNA, 22 bp, Glycine max
ggctgtgttgagggtggaggat

SEQ ID NO: 72 - DNA, 44 bp, Glycine max
gaaggtgaccaagttcatgctgatccaaacccaacgtaacctgg

SEQ ID NO: 73 - DNA, 45 bp, Glycine max
gaaggtcggagtcaacggattagatccaaacccaacgtaacctga

SEQ ID NO: 74 - DNA, 401 bp, Glycine max
ttattgtatggggagaattgcacaccagaccagacaagggacctaaacctaaccctgtgaactgaaaatggtgtgaggaagaattgggatcgaaatggtaaaaaggaaaagaaaagatataaatatattattataaaattggtaaagaaaaggagaagaggaaaggaaggctgtgttgagggtggaggataggcacgaagycaggttacgttgggtttggatctgatggagatggtggattgtggaccccactccaccccaaagccaatctctttttctgtttttcttctttcgcagactcccatatggacctgggttttacagatgggcattggcccaacaactatctactatatccacttcccctatttgcttatccactccccttttcaataaaacat

SEQ ID NO: 75 - DNA, 29 bp, Glycine max
atgagaagaaatggactgatggaagtgtt

SEQ ID NO: 76 - DNA, 44 bp, Glycine max
gaaggtgaccaagttcatgctcaagccttgcaaggttcccagaa

SEQ ID NO: 77 - DNA, 43 bp, Glycine max
gaaggtcggagtcaacggattaagccttgcaaggttcccagac

SEQ ID NO: 78 - DNA, 121 bp, Glycine max
ccaaaagcattattaaaatttcaatatccatacctttccaagccttgcaaggttcccagamacagcatctaatggaacacttccatcagtccatttcttctcatgaatggtgatacccatt

SEQ ID NO: 79 - DNA, 26 bp, Glycine max
ccatttctgaatcaacagacgcccaa

SEQ ID NO: 80 - DNA, 50 bp, Glycine max
gaaggtgaccaagttcatgctggaaaatcattgttagttacctgatctag

SEQ ID NO: 81 - DNA, 50 bp, Glycine max
gaaggtcggagtcaacggattggaaaatcattgttagttacctgatctac

SEQ ID NO: 82 - DNA, 401 bp, Glycine max
tgtccttcccactcttgagttaaattcttgtgatctttacacaaaaacctgagcaagaaacattagcccaaaatgaatccaacaaggatgcacccaccagacttgaaaatgatacaaggtttggaaagaatttggtttacacaagaagatcaaaggccatttctgaatcaacagacgcccaagaggccaatccaacwccgstagatcaggtaactaacaatgattttccaattttgaatgataatcttttagcttctcctaatgaaacggaaatttcagaacatattgatgacctttatcttcccatttcctttagaaaaggaaccagaacatgtgccaaaaagcctctctatcctctctcaaatctcatttcataaattttttccaacccataaaacctt

SEQ ID NO: 83 - DNA, 30 bp, Glycine max
aatcttgattcctatttgggtttcctwgta

SEQ ID NO: 84 - DNA, 48 bp, Glycine max
gaaggtgaccaagttcatgctaatatggggtttaaacagctactcata

SEQ ID NO: 85 - DNA, 50 bp, Glycine max
gaaggtcggagtcaacggattctaatatggggtttaaacagctactcatt

SEQ ID NO: 86 - DNA, 401 bp, Glycine max
ccaaatgaaatctttgcaaattttctcaatgtctttacacaccaagactggcaacttggacgcttgaagcacctatgcaggaatattaaacaagcaagcttgtgccaaagttaccctgccagtcgtggataggatcttagctttccagctagtaatttttcgccagatatttctaatatggggtttaaacagctactcatwtctcttatgatcctatacwaggaaacccaaataggaatcaagattattagtaatagtcacctgaagctgttgactaagctccctcaccttagcttatgagacattagttragaagagaacctttgatttgctcatgttgatcttctggctagatgcattagcaaaagggtttaatatctggttgacttccccagctgcta

SEQ ID NO: 87 - DNA, 20 bp, Glycine max
aaagaccagcactccagcat

SEQ ID NO: 88 - DNA, 20 bp, Glycine max
tagaggaaagggtggtggtg

SEQ ID NO: 89 - DNA, 16 bp, Glycine max
tagatcctccattttt

SEQ ID NO: 90 - DNA, 15 bp, Glycine max
atcctccttttttcc

SEQ ID NO: 91 - DNA, 842 bp, Glycine max
  misc_feature (1)..(842): n = A, C, G, or T
tgcgggctaatgttgtgctgtgtgtgagggtggggtcgatgagatttagattcnnnnnnntaagctactgtcaagaatacrtaaaacgtttagatcttaaaacacaagaaagaccagcactccagcatgcagtcaccactgtactcaatgtttagatcctccwtttttcccgtggccatgtccacctcttcctccaccaccaccctttcctctatgatgtttttttgctccacctaatgccctgttagcatcaggtctcacttgcatctcttgtggcaagggaagaatatgatccacacatttacggaagctaaaatcatcgacagattcatcgtccctgacgaggaagaagcacctacttttccacatagggtgcttgcggacttgaaaaacacggattcctcctccaatcttagtatcaggttctgtgtgaccgttcttaagcaactccagtagcatcatatgttcatactgccacacaaaaaacaactcatagtgaattcagcaagtagattaaggttgtgggcatcatcaagacacgttactaagactaagccaatttaaacattaactggccatgaatttgcaygcagttgaatcttgaattgcacatgcattaaaatagattacaaaccattttatatcacatgttgatggcactgaaagccatttcacaagctcatgcattaaaataattgtttcctaataatcctatgcacttctggaaatctttgcagtgaatctgccaaacatgataaataaatagattgtraaaaagagagtttgcgtacatatccaaccaagctattcactaaatttagccggraaaaatatttcaaaaa

SEQ ID NO: 92 - DNA, 20 bp, Glycine max
aaagaccagcactccagcat

SEQ ID NO: 93 - DNA, 20 bp, Glycine max
tagaggaaagggtggtggtg

SEQ ID NO: 94 - DNA, 19 bp, Glycine max
atgtttagatcctccattt

SEQ ID NO: 95 - DNA, 18 bp, Glycine max
atgtttagatcctccttt

SEQ ID NO: 96 - DNA, 401 bp, Glycine max
aaaagtaggtgcttcttcctcgtcagggacgatgaatctgtcgatgattttagcttccgtaaatgtgtggatcatattcttcccttgccacaagagatgcaagtgagacctgatgctaacagggcattaggtggagcaaaaaaacatcatagaggaaagggtggtggtggaggaagaggtggacatggccacgggaaaaawggaggatctaaacattgagtacagtggtgactgcatgctggagtgctggtctttcttgtgttttaagatctaaacgttttacgtattcttgacagtagcttatttcatgaatctaaatctcatcgaccccaccctcacacacagcacaacattagcccgcatgacctgcaaagtttctttatgtaattattttgagtttt

SEQ ID NO: 97 - DNA, 19 bp, Glycine max
tgcacacacacccaatcac

SEQ ID NO: 98 - DNA, 20 bp, Glycine max
accttctaatcccgcctctt

SEQ ID NO: 99 - DNA, 18 bp, Glycine max
aagagagaatccagttga

SEQ ID NO: 100 - DNA, 19 bp, Glycine max
aaaagagagaatccaattg

SEQ ID NO: 101 - DNA, 630 bp, Glycine max
aggaatwagataattatgactgccaggcaagtatcaggcaccctaggagcacaagtcgtcacctttacataaactattaactaacatttcaactaaaactactcgccgtttattaaatccataacaacacgtttgaggcttccattgtttttctcaagcagtctcttattcatctctttgtcacgaaaaccctgcacacacacccaatcactaatcaaaagagagaatccarttgaacaaattgcttagtaagaggcgggattagaaggtaaagaacttcaaattgcaggcattttgataataaaataaaaggtgaacaaccagaacaacttaccatctcctgtaactcttcaagcatatggtcccactcagaaacaccacagacatcaacaccacagagagcgtctagggattggtccagatcgtactcattcttcctcaagatctccttgttcaaatcaacctgtttaaaacccatctcctcaagctctttaagtagattctcctccacagaattaattcctccaccagtacccatagatgacgatggcacatccacggttggggattgctgattagatggaacagctggggttgtttcagacagatcaatcatggtcatagcykktt

SEQ ID NO: 102 - DNA, 20 bp, Glycine max
agatcaatggcacccttacg

SEQ ID NO: 103 - DNA, 23 bp, Glycine max
ttccagatccagatagcaacttc

SEQ ID NO: 104 - DNA, 17 bp, Glycine max
tccagtgacttttgcac

SEQ ID NO: 105 - DNA, 18 bp, Glycine max
ttccagtgacttttgtac

SEQ ID NO: 106 - DNA, 401 bp, Glycine max
tctatgagtgtgattaacttggccttccattcaaaaatttcctttttctttttcttttatagatatggtttaagttttgttcttttaaatgtgcaataggtggcatccaggaaaagatcaatggcacccttacgtgagcyttatagcttkgttactgtcaaggattttgctaaagcctttcagatttccagtgacttttgyacwgtcaaaggatcctcaaattaccaataaccccttgtagaagttgctatctggatctggaaatgattcttttattacgattgtttaaaaaaattgtattgctacttctcttgcggatgatgcataaattattgggtttatgtacttgtatttctgtttattgtgtactcgttccttttatgtgctctctaaaatcatat

SEQ ID NO: 107 - DNA, 25 bp, Glycine max
cagaaagaagagcaccaccaaccaa

SEQ ID NO: 108 - DNA, 44 bp, Glycine max
gaaggtgaccaagttcatgctatccactccctttcctgttccta

SEQ ID NO: 109 - DNA, 42 bp, Glycine max
gaaggtcggagtcaacggattccactccctttcctgttcctg

SEQ ID NO: 110 - DNA, 401 bp, Glycine max
ggtcagactcaacagttattggttgactgaccatatccaagaaaatgatgttcactaaccttaaagtgaagccatcattcataaaatagcttattagtcatttcatcactatagtaaccctgcactaaatcattctcattaccagcacttcaaattactcatttccataattccgttgatccactccctttcctgttcctrccatggccctgattggttggtggtgctcttctttctgctttccttcaggttgcggttgacaggcttgcttctcgtcaagttcttgacttcttttgtgcaagaaaactcgatgagatgctgctcggcaagttgaacatgaagctgctgtccattgatgctctggctgatgatgcagatcaaaagcagttcagagatccacg

SEQ ID NO: 111 - DNA, 19 bp, Glycine max
tggctggttcgtacaatcg

SEQ ID NO: 112 - DNA, 22 bp, Glycine max
ggagcgaggtcaaagagaagta

SEQ ID NO: 113 - DNA, 16 bp, Glycine max
tctcatctcaattcaa

SEQ ID NO: 114 - DNA, 15 bp, Glycine max
tctcatcacaattca

SEQ ID NO: 115 - DNA, 401 bp, Glycine max
ctccgccgccgtcggcgtcgccctctcctacctctccttcggcgtctcctccaacctccacttcctcgtccccatgttcctcggctacgcctccatgctcctctttcgtccccgatgcggcatcctcaccttcttcctcggattcggctacctcattggctggttcgtacaatcgagatctattttgtcataatctcatcwcaattcaattctacttctctttgacctcgctccgcgtttgcgtttctctcttctgcagccacgtgtattacatgagcggtgacgcatggaaggaaggtggcatcgatgccactggtaagcgccaattaattatttatttattttttgaattgaggaatgagggaattgactgattagtagtatttcactggtaacgttat

SEQ ID NO: 116 - DNA, 18 bp, Glycine max
caagccgacatcggaaaa

SEQ ID NO: 117 - DNA, 20 bp, Glycine max
cgacattgtccagggctatt

SEQ ID NO: 118 - DNA, 15 bp, Glycine max
caacgtcacttgaaa

SEQ ID NO: 119 - DNA, 14 bp, Glycine max
acgtcgcttgaaaa

SEQ ID NO: 120 - DNA, 401 bp, Glycine max
gggttatttttcagttgacgtcggctaggttttttttggtcaagattagccaatgatgtttttttggctgacatcgaccgatcatgttttttgccgacattgtccagggctattttyggctgatatcggctaggatattttctggtcaatgttagctagtgatgctttttggttgacatcaactaaaactatttttcaagygacgttgatcagagctatttttttccgatgtcggcttgggtcattggcaccaacaaaaaatagcctcgatcaaagttagccaaaaaaatcctagtcgatgtcggccaaaaaaatagtcatggccgatgttggccaaaaaacatcatcgcttgacgtcggccaaaaagacccttgctggcatcggttaaaatagtctcggt

SEQ ID NO: 121 - DNA, 23 bp, Glycine max
tccggtatcggtttataagtttg

SEQ ID NO: 122 - DNA, 20 bp, Glycine max
acgattgtgctgaagtgctg

SEQ ID NO: 123 - DNA, 16 bp, Glycine max
ctgatccaatccaaac

SEQ ID NO: 124 - DNA, 16 bp, Glycine max
ctgatccaaaccaaac

SEQ ID NO: 125 - DNA, 401 bp, Glycine max
aaaaaataaattaraaaaaaaaaycaattttacttgtaagttttaattagatcgactactattatgttttggttccrataatcagtttcagttcacttttttttacagcaaatttcggttcactttatggttatctaacctgatattattcgattccggtatcggtttataagtttgcaaacccgaataatctgatccaawccaaacgcawatacactgaactttcgttctagagctctgcagcacttcagcacaatcgtttttattttttctcagggtaccagagcttctgccatcatggatggacaccgtgttactccaaaaatacaagattgatttgatgattgaaaaataagaatttaaacataaaggaaaaaagagaagtctcaagattaaaattc

SEQ ID NO: 126 - DNA, 18 bp, Glycine max
cgtccagccacacacaac

SEQ ID NO: 127 - DNA, 24 bp, Glycine max
gaaatcatcaacaagtgatcatcc

SEQ ID NO: 128 - DNA, 17 bp, Glycine max
aattgcttcacgtttca

SEQ ID NO: 129 - DNA, 16 bp, Glycine max
ttgcttcaggtttcat

SEQ ID NO: 130 - DNA, 401 bp, Glycine max
acacgtacataagttattaaatttaatttaagagaaaataattcataatatttaattatttttcatttaaagattgattgaaatcatcaacaagtgatcatccrtgtaataattttttgcatattgaatwtagtgactcaaaaagtttcgataaakataggattagtttcattwgggtgcttatataaagatcatgaaacstgaagcaattctgttgtgtgtggctggacgatcatggtccggacgaccggggcagacgagggaaattgtactcagttagttggttaatttctgttataacttccataacagaaataattattttctgttagagtctatgggtataaatacattgtatttatcaactcttgtacgttcatgattgattaatgcataagtcc

SEQ ID NO: 131 - DNA, 24 bp, Glycine max
ttttaggtttgactgatcttggaa

SEQ ID NO: 132 - DNA, 21 bp, Glycine max
aatttctttgccatgcaagtg

SEQ ID NO: 133 - DNA, 16 bp, Glycine max
tagaactcagagaccc

SEQ ID NO: 134 - DNA, 18 bp, Glycine max
attagaacttagagaccc

SEQ ID NO: 135 - DNA, 401 bp, Glycine max
tgattaagcccaagtacaacataaaaaaaaaatacaaaataacaaatgtattggattgcgctcgccccccagtgatcttatctctggtgatttcggcctcctattagcttcttaaccatggttgtaatcctaatcactcttctccctatgaatttctttgccatgcaagtgcaataaaaaccttcccaatttgggtctctragttctaatcctttccaagatcagtcaaacctaaaatgaaacaagacagttacaccataacaagtcctagaatggaacacgtcaacggaaaaacacaatgaaattacactaaaaagagaagaaaaagcatggaaacctaactagacaatatagagagatgacatgagatgcaacgattaaacaactagatcggtgatgcc

SEQ ID NO: 136 - DNA, 24 bp, Glycine max
agctgtggcttactaacattaggg

SEQ ID NO: 137 - DNA, 26 bp, Glycine max
atttaaacctatccaaatcaactacg

SEQ ID NO: 138 - DNA, 16 bp, Glycine max
cttgcaaacttggatc

SEQ ID NO: 139 - DNA, 15 bp, Glycine max
cttgcaaacctggat

SEQ ID NO: 140 - DNA, 401 bp, Glycine max
atgtttgattcccaattcaaccggtgtaatcggtcgatcggagctagctagagtctgataacatggtagttataacttacatctatttattttttagaaaaaaataaactagtaagttataactacaattgtcttaaaatatacttaagctgtggcttactaacattaggggtgagcatggctcaatccggcttgcaaacytggatccactcgtagttgatttggataggtttaaatttttaaattagacgatgtattttttaggatgagtttgagttaagttttgagtaatctcaaactagtttttttgtcttaataagtagagcttgataaaaaaaaaaaaaaatatatatatatatatatatatatatatatatatatatatatatttatcaaagatt

SEQ ID NO: 141 - DNA, 17 bp, Glycine max
caatggccgacatccac

SEQ ID NO: 142 - DNA, 26 bp, Glycine max
aacaatgcatgtgatagaataaaagc

SEQ ID NO: 143 - DNA, 13 bp, Glycine max
caccacattcaac

SEQ ID NO: 144 - DNA, 13 bp, Glycine max
caccacatccaac

SEQ ID NO: 145 - DNA, 121 bp, Glycine max
gtcacccccacataaatcaaactttacaatattctagtcaatggccgacatccaccacatycaacataaataggttttgrcgttgcttttattctatcacatgcattgttcaaagctaaga

(19) United States
(12) Patent Application Publication
(10) Pub. No.: US 2014/0189902 A1
(43) Pub. Date: Jul. 3, 2014

(54) Compositions Associated with Soybean Iron Deficiency Tolerance and Methods of Use

(71) Applicant: PIONEER HI BRED INTERNATIONAL INC., Johnston, IA (US)

(72) Inventors: Julian M. Chaky, Urbandale, IA (US); Martin A. Fabrizius, Redwood Falls, MN (US); David L. Hyten, JR., Johnston, IA (US); Nadejda N. Krasheninnik, Fargo, ND (US); Jordan Spear, Algona, IA (US); John B. Woodward, Ankeny, IA (US); Yanwen Xiong, Johnston, IA (US)

(73) Assignee: PIONEER HI BRED INTERNATIONAL INC., Johnston, IA (US)

(21) Appl. No.: 13/798,409

(22) Filed: Mar. 13, 2013

Related U.S. Application Data
(60) Provisional application No. 61/746,340, filed on Dec. 27, 2012.

(51) Int. Cl. C12Q 1/68

(52) U.S. Cl. CPC C12Q 1/6881
USPC 800/260; 536/24.3; 435/6.11

(57) ABSTRACT

Molecular markers useful for identifying, selecting, and/or providing soybean plants displaying tolerance, improved tolerance, or susceptibility to iron deficiency, methods of their use, and compositions having one or more marker loci are provided. Methods comprise detecting at least one marker locus, detecting a haplotype, and/or detecting a marker profile. Methods may further comprise crossing a selected soybean plant with a second soybean plant. Isolated polynucleotides, primers, probes, kits, systems, as well as soybean plants, seeds, and parts thereof are also provided.

### REFERENCE TO SEQUENCE LISTING SUBMITTED ELECTRONICALLY

The official copy of the sequence listing is submitted electronically via EFS-Web as an ASCII formatted sequence listing with a file named “4684.seqlist_ST25.txt” created on Mar. 1, 2013, and having a size of 38 kilobytes and is filed concurrently with the specification. The sequence listing contained in this ASCII formatted document is part of the specification and is herein incorporated by reference in its entirety.

### FIELD OF THE INVENTION

This invention relates to compositions useful for identifying iron deficiency tolerant or susceptible soybean plants and methods of their use.

### BACKGROUND

Soybeans (Glycine max L. Merr.) are a major cash crop and investment commodity in North America and elsewhere. Soybean is the world's primary source of seed oil and seed protein. Improving soybean tolerance to diverse and/or adverse growth conditions is crucial for maximizing yields. Studies have shown that even mild IDC symptoms are an indication that yield is being negatively affected (Fehr (1982) J Plant Nutr 5:611-621).

Iron-deficiency chlorosis (IDC, or FEC), reduces soybean yields. Iron is required for the synthesis of chlorophyll and, although the amount of iron is sufficient in most soils, it is often in an insoluble form that cannot be used by the plant. Iron deficiency is typically associated with soils having high pH, high salt content, cool temperatures or other environmental factors that decrease iron solubility. Chlorosis develops due to a lack of chlorophyll in the leaves of affected plants, manifesting as yellowing of the leaves.

There remains a need for soybean plants with improved tolerance to iron deficiency and methods for identifying, selecting and providing such plants, including improved markers for identifying plants possessing tolerance or susceptibility.

### SUMMARY

Molecular markers useful for identifying, selecting, and/or providing soybean plants displaying tolerance, improved tolerance, or susceptibility to iron deficiency, methods of their use, and compositions having one or more marker loci are provided. Methods comprise detecting at least one marker locus, detecting a haplotype, and/or detecting a marker profile. Methods may further comprise crossing a selected soybean plant with a second soybean plant. Isolated polynucleotides, primers, probes, kits, systems, etc., are also provided.

### BRIEF DESCRIPTION OF DRAWINGS

FIG. 1 (parts A-D) provides an exemplary genetic map for at least a portion of linkage group A1 (chromosome 5).

FIG. 2 illustrates an apparent misassembly for chromosome 5 (FIG. 2A) and correction based on mapping data (FIG. 2B).

### SUMMARY OF SEQUENCES

SEQ ID NOs: 1-5 comprise nucleotide sequences of regions of the soybean genome, each capable of being used as a probe or primer, either alone or in combination, for the detection of marker locus S00405 on LG-A1 (G. max chromosome 5 (Gm05)). In certain examples, SEQ ID NOs: 1 and 2 are used as allele specific primers and SEQ ID NOs: 3 and 4 are used as allele probes. SEQ ID NO: 5 is the genomic DNA region encompassing marker locus S00405.

SEQ ID NOs: 6-10 comprise nucleotide sequences of regions of the soybean genome, each capable of being used as a probe or primer, either alone or in combination, for the detection of marker locus S15121 on LG-A1. In certain examples, SEQ ID NOs: 5 and 6 are used as allele specific primers and SEQ ID NOs: 7 and 8 are used as allele probes. SEQ ID NO: 10 is the genomic DNA region encompassing marker locus S15121.

SEQ ID NOs: 11-15 comprise nucleotide sequences of regions of the soybean genome, each capable of being used as a probe or primer, either alone or in combination, for the detection of marker locus S15124 on LG-A1. In certain examples, SEQ ID NOs: 9 and 10 are used as allele specific primers and SEQ ID NOs: 11 and 12 are used as allele probes. SEQ ID NO: 15 is the genomic DNA region encompassing marker locus S15124.

SEQ ID NOs: 16-20 comprise nucleotide sequences of regions of the soybean genome, each capable of being used as a probe or primer, either alone or in combination, for the detection of marker locus S04776 on LG-A1. In certain examples, SEQ ID NOs: 13 and 14 are used as allele specific primers and SEQ ID NOs: 15 and 16 are used as allele probes. SEQ ID NO: 20 is the genomic DNA region encompassing marker locus S04776.

SEQ ID NOs: 21-25 comprise nucleotide sequences of regions of the soybean genome, each capable of being used as a probe or primer, either alone or in combination, for the detection of marker locus S15081 on LG-A1. In certain examples, SEQ ID NOs: 21 and 22 are used as allele specific primers and SEQ ID NOs: 23 and 24 are used as allele probes. SEQ ID NO: 25 is the genomic DNA region encompassing marker locus S15081.

SEQ ID NOs: 26-29 comprise nucleotide sequences of regions of the soybean genome, each capable of being used as a probe or primer, either alone or in combination, for the detection of marker locus S05017 on LG-A1. In certain examples, SEQ ID NO: 26 is used as a allele specific primer and SEQ ID NOs: 27 and 28 are used as allele probes. SEQ ID NO: 29 is the genomic DNA region encompassing marker locus S05017.

SEQ ID NOs: 30-33 comprise nucleotide sequences of regions of the soybean genome, each capable of being used as a probe or primer, either alone or in combination, for the detection of marker locus S07022 on LG-A1. In certain examples, SEQ ID NO: 30 is used as a allele specific primer and SEQ ID NOs: 31 and 32 are used as allele probes. SEQ ID NO: 33 is the genomic DNA region encompassing marker locus S07022.

SEQ ID NOs: 34-37 comprise nucleotide sequences of regions of the soybean genome, each capable of being used as a probe or primer, either alone or in combination, for the detection of marker locus S10456 on LG-A1. In certain examples, SEQ ID NO: 34 is used as a allele specific primer and SEQ ID NOs: 35 and 36 are used as allele probes. SEQ ID NO: 37 is the genomic DNA region encompassing marker locus S10456.

SEQ ID NOs: 38-42 comprise nucleotide sequences of regions of the soybean genome, each capable of being used as a probe or primer, either alone or in combination, for the detection of marker locus S15126 on LG-A1. In certain examples, SEQ ID NOs: 38 and 39 are used as allele specific primers and SEQ ID NOs: 40 and 41 are used as allele probes. SEQ ID NO: 42 is the genomic DNA region encompassing marker locus S15126.

SEQ ID NOs: 43-47 comprise nucleotide sequences of regions of the soybean genome, each capable of being used as a probe or primer, either alone or in combination, for the detection of marker locus S15071 on LG-A1. In certain examples, SEQ ID NOs: 43 and 44 are used as allele specific primers and SEQ ID NOs: 45 and 46 are used as allele probes. SEQ ID NO: 47 is the genomic DNA region encompassing marker locus S15071.

SEQ ID NOs: 48-52 comprise nucleotide sequences of regions of the soybean genome, each capable of being used as a probe or primer, either alone or in combination, for the detection of marker locus S15122 on LG-A1. In certain examples, SEQ ID NOs: 48 and 49 are used as allele specific primers and SEQ ID NOs: 50 and 51 are used as allele probes. SEQ ID NO: 52 is the genomic DNA region encompassing marker locus S15122.

SEQ ID NOs: 53-56 comprise nucleotide sequences of regions of the soybean genome, each capable of being used as a probe or primer, either alone or in combination, for the detection of marker locus S13062 on LG-A1. In certain examples, SEQ ID NO: 53 is used as a allele specific primer and SEQ ID NOs: 54 and 55 are used as allele probes. SEQ ID NO: 56 is the genomic DNA region encompassing marker locus S13062.

SEQ ID NOs: 57-61 comprise nucleotide sequences of regions of the soybean genome, each capable of being used as a probe or primer, either alone or in combination, for the detection of marker locus S15125 on LG-A1. In certain examples, SEQ ID NOs: 57 and 58 are used as allele specific primers and SEQ ID NOs: 59 and 60 are used as allele probes. SEQ ID NO: 61 is the genomic DNA region encompassing marker locus S15125.

SEQ ID NOs: 62-66 comprise nucleotide sequences of regions of the soybean genome, each capable of being used as a probe or primer, either alone or in combination, for the detection of marker locus S15123 on LG-A1. In certain examples, SEQ ID NOs: 62 and 63 are used as allele specific primers and SEQ ID NOs: 64 and 65 are used as allele probes. SEQ ID NO: 66 is the genomic DNA region encompassing marker locus S15123.

SEQ ID NOs: 67-70 comprise nucleotide sequences of regions of the soybean genome, each capable of being used as a probe or primer, either alone or in combination, for the detection of marker locus S12985 on LG-A1. In certain examples, SEQ ID NO: 67 is used as a allele specific primer and SEQ ID NOs: 68 and 69 are used as allele probes. SEQ ID NO: 70 is the genomic DNA region encompassing marker locus S12985.

SEQ ID NOs: 71-74 comprise nucleotide sequences of regions of the soybean genome, each capable of being used as a probe or primer, either alone or in combination, for the detection of marker locus S13064 on LG-A1. In certain examples, SEQ ID NO: 71 is used as a allele specific primer and SEQ ID NOs: 72 and 73 are used as allele probes. SEQ ID NO: 74 is the genomic DNA region encompassing marker locus S13064.

SEQ ID NOs: 75-78 comprise nucleotide sequences of regions of the soybean genome, each capable of being used as a probe or primer, either alone or in combination, for the detection of marker locus S05933 on LG-A1. In certain examples, SEQ ID NO: 75 is used as a allele specific primer and SEQ ID NOs: 76 and 77 are used as allele probes. SEQ ID NO: 78 is the genomic DNA region encompassing marker locus S05933.

SEQ ID NOs: 79-82 comprise nucleotide sequences of regions of the soybean genome, each capable of being used as a probe or primer, either alone or in combination, for the detection of marker locus S13078 on LG-A1. In certain examples, SEQ ID NO: 79 is used as a allele specific primer and SEQ ID NOs: 80 and 81 are used as allele probes. SEQ ID NO: 82 is the genomic DNA region encompassing marker locus S13078.

SEQ ID NOs: 83-86 comprise nucleotide sequences of regions of the soybean genome, each capable of being used as a probe or primer, either alone or in combination, for the detection of marker locus S13073 on LG-A1. In certain examples, SEQ ID NO: 83 is used as a allele specific primer and SEQ ID NOs: 84 and 85 are used as allele probes. SEQ ID NO: 86 is the genomic DNA region encompassing marker locus S13073.

SEQ ID NOs: 87-91 comprise nucleotide sequences of regions of the soybean genome, each capable of being used as a probe or primer, either alone or in combination, for the detection of marker locus S01261 on LG-A1. In certain examples, SEQ ID NOs: 87 and 88 are used as allele specific primers and SEQ ID NOs: 89 and 90 are used as allele probes. SEQ ID NO: 91 is the genomic DNA region encompassing marker locus S01261.

SEQ ID NOs: 92-96 comprise nucleotide sequences of regions of the soybean genome, each capable of being used as a probe or primer, either alone or in combination, for the detection of marker locus S14531 on LG-A1. In certain examples, SEQ ID NOs: 92 and 93 are used as allele specific primers and SEQ ID NOs: 94 and 95 are used as allele probes. SEQ ID NO: 96 is the genomic DNA region encompassing marker locus S14531.

SEQ ID NOs: 97-101 comprise nucleotide sequences of regions of the soybean genome, each capable of being used as a probe or primer, either alone or in combination, for the detection of marker locus S01282 on LG-A1. In certain examples, SEQ ID NOs: 97 and 98 are used as allele specific primers and SEQ ID NOs: 99 and 100 are used as allele probes. SEQ ID NO: 101 is the genomic DNA region encompassing marker locus S01282.

SEQ ID NOs: 102-106 comprise nucleotide sequences of regions of the soybean genome, each capable of being used as a probe or primer, either alone or in combination, for the detection of marker locus S14582 on LG-A1. In certain examples, SEQ ID NOs: 102 and 103 are used as allele specific primers and SEQ ID NOs: 104 and 105 are used as allele probes. SEQ ID NO: 106 is the genomic DNA region encompassing marker locus S14582.

SEQ ID NOs: 107-110 comprise nucleotide sequences of regions of the soybean genome, each capable of being used as a probe or primer, either alone or in combination, for the detection of marker locus S10245 on LG-A1. In certain examples, SEQ ID NO: 107 is used as a allele specific primer and SEQ ID NOs: 108 and 109 are used as allele probes. SEQ ID NO: 110 is the genomic DNA region encompassing marker locus S10245.

SEQ ID NOs: 111-115 comprise nucleotide sequences of regions of the soybean genome, each capable of being used as a probe or primer, either alone or in combination, for the detection of marker locus S14581 on LG-A1. In certain examples, SEQ ID NOs: 111 and 112 are used as allele specific primers and SEQ ID NOs: 113 and 114 are used as allele probes. SEQ ID NO: 115 is the genomic DNA region encompassing marker locus S14581.

SEQ ID NOs: 116-120 comprise nucleotide sequences of regions of the soybean genome, each capable of being used as a probe or primer, either alone or in combination, for the detection of marker locus S10446 on LG-A1. In certain examples, SEQ ID NOs: 116 and 117 are used as allele specific primers and SEQ ID NOs: 118 and 119 are used as allele probes. SEQ ID NO: 120 is the genomic DNA region encompassing marker locus S10446.

SEQ ID NOs: 121-125 comprise nucleotide sequences of regions of the soybean genome, each capable of being used as a probe or primer, either alone or in combination, for the detection of marker locus S14561 on LG-A1. In certain examples, SEQ ID NOs: 121 and 122 are used as allele specific primers and SEQ ID NOs: 123 and 124 are used as allele probes. SEQ ID NO: 125 is the genomic DNA region encompassing marker locus S14561.

SEQ ID NOs: 126-130 comprise nucleotide sequences of regions of the soybean genome, each capable of being used as a probe or primer, either alone or in combination, for the detection of marker locus S14552 on LG-A1. In certain examples, SEQ ID NOs: 126 and 127 are used as allele specific primers and SEQ ID NOs: 128 and 129 are used as allele probes. SEQ ID NO: 130 is the genomic DNA region encompassing marker locus S14552.

SEQ ID NOs: 131-135 comprise nucleotide sequences of regions of the soybean genome, each capable of being used as a probe or primer, either alone or in combination, for the detection of marker locus S14562 on LG-A1. In certain examples, SEQ ID NOs: 131 and 132 are used as allele specific primers and SEQ ID NOs: 133 and 134 are used as allele probes. SEQ ID NO: 135 is the genomic DNA region encompassing marker locus S14562.

SEQ ID NOs: 136-140 comprise nucleotide sequences of regions of the soybean genome, each capable of being used as a probe or primer, either alone or in combination, for the detection of marker locus S13012 on LG-A1. In certain examples, SEQ ID NOs: 136 and 137 are used as allele specific primers and SEQ ID NOs: 138 and 139 are used as allele probes. SEQ ID NO: 140 is the genomic DNA region encompassing marker locus S13012.

SEQ ID NOs: 141-145 comprise nucleotide sequences of regions of the soybean genome, each capable of being used as a probe or primer, either alone or in combination, for the detection of marker locus S05107 on LG-A1. In certain examples, SEQ ID NOs: 141 and 142 are used as allele specific primers and SEQ ID NOs: 143 and 144 are used as allele probes. SEQ ID NO: 145 is the genomic DNA region encompassing marker locus S05107.

### DETAILED DESCRIPTION

Method for identifying a soybean plant or germplasm that displays tolerance, improved tolerance, or susceptibility to iron deficiency, the method comprising detecting at least one allele of one or more marker loci associated with iron deficiency tolerance are provided.

In some examples, the method involves detecting a single marker locus associated with iron deficiency tolerance in soybean. In some examples the method comprises detecting a polymorphism flanked by and including a marker locus from 0 cM to 30 cM on LG A1. In some examples the method comprises detecting a polymorphism from about 0-25 cM, 0-20 cM, 0-15 cM, 0-10 cM, 0-5 cM, or about 0-2.5 cM on LG A1. In some examples the method comprises detecting a polymorphism linked to a marker locus selected from the group consisting of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107. In some examples the method comprises detecting a polymorphism closely linked to a marker locus selected from the group consisting of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107. In some examples the method comprises detecting a polymorphism in a marker locus selected from the group consisting of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107. In some examples, the method comprises detecting a polymorphism using a marker selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, S14581-001-Q001, S10446-001-Q1, S14561-001-Q001, S14552-001-Q001, S14562-001-Q001, S13012-001-Q002, and 505107-001-Q002.

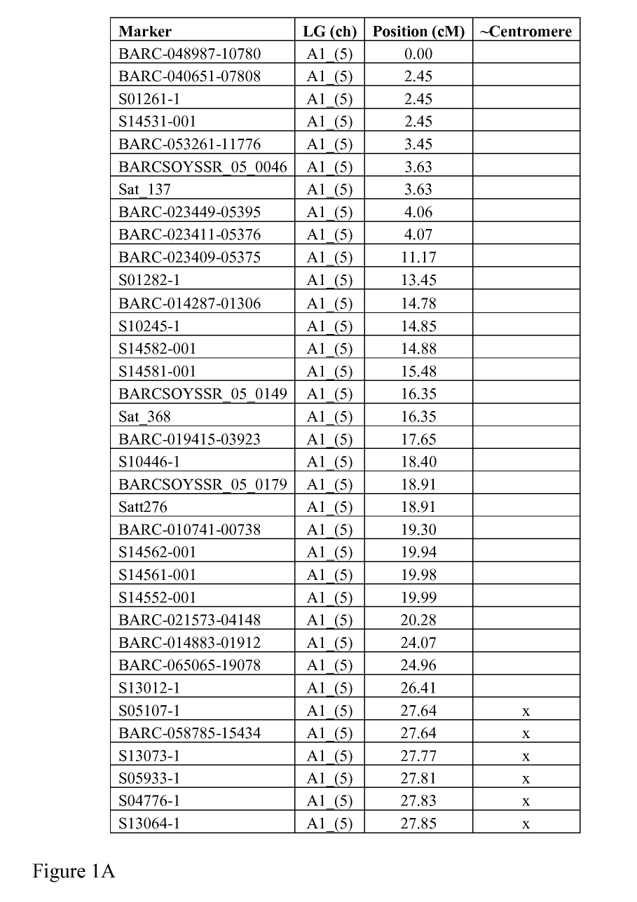

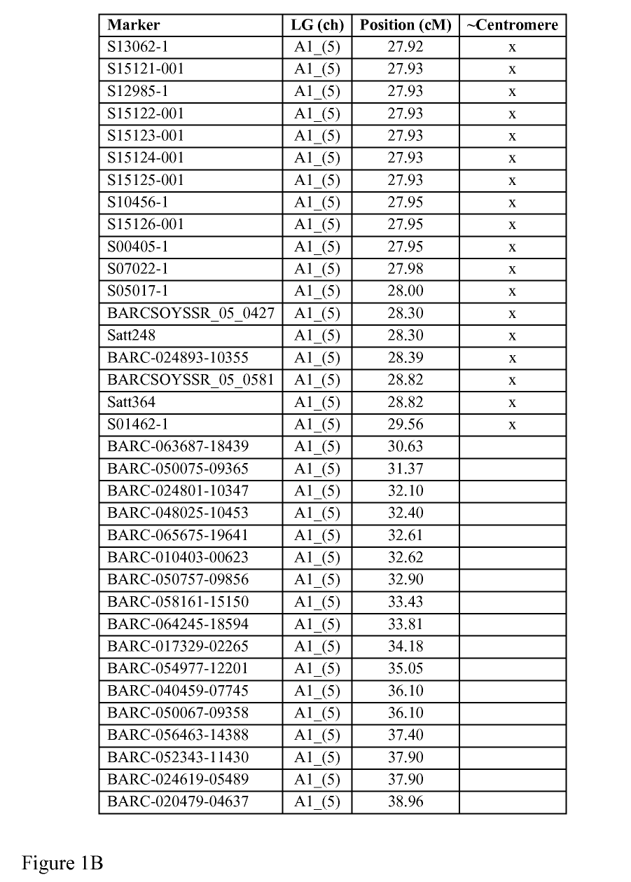

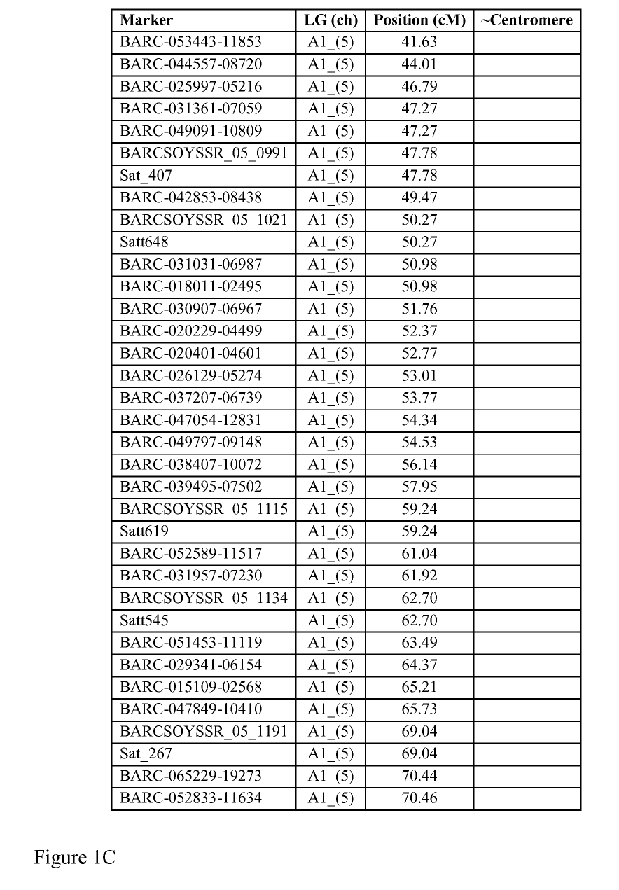

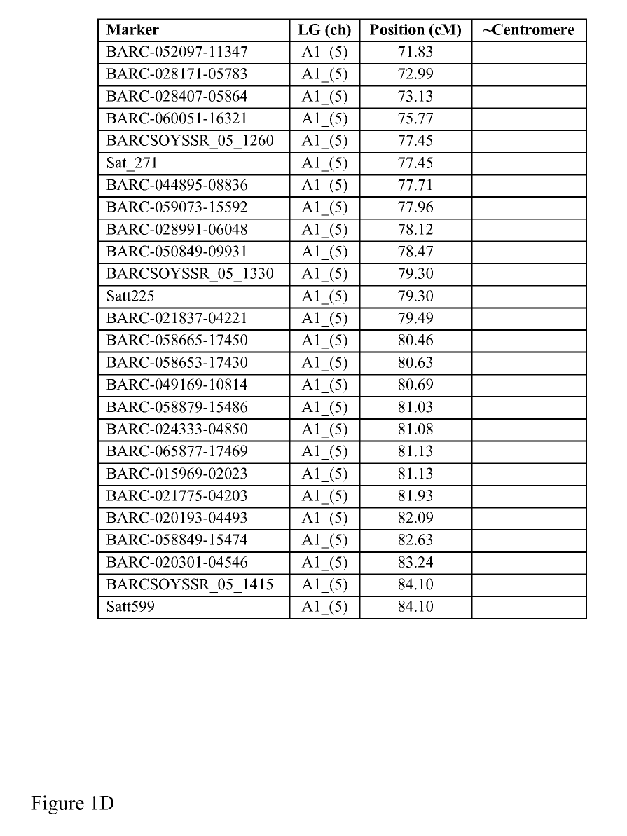

In other examples, the method involves detecting a haplotype comprising two or more marker loci, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 marker loci, or more. In certain examples, the haplotype comprises two or more markers selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, S14581-001-Q001, S10446-001-Q1, S14561-001-Q001, S14552-001-Q001, S14562-001-Q001, S13012-001-Q002, and S05107-001-Q002. In further examples, the haplotype comprises markers from the set of markers described in FIG. 1, or the set of marker described in Table 6.

In some examples, the one or more alleles are favorable alleles that positively correlate with tolerance or improved tolerance to iron deficiency. In other examples, the one or more alleles are disfavored alleles that positively correlate with susceptibility or increased susceptibility to iron deficiency.

In certain examples, the one or more marker locus detected comprises one or more markers on LG-A1 selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, 514581-001-Q001, S10446-001-Q1, S14561-001-Q001, S14552-001-Q001, S14562-001-Q001, S13012-001-Q002, and S05107-001-Q002. In other examples, the one or more marker locus detected comprises one or more markers within the chromosome interval on linkage group A1 flanked by and including S15081-001 (8712346 bp, 27.94 cM) and S01282-1-A (2012649 bp, 13.45 cM), or an interval flanked by and including BARC-044481-08709 (9097270 bp, 22.52 cM) and BARC-019031-03052 (7546740 bp, 14.63 cM), or an interval flanked by and including the top of LG A1 (0 cM) and Sat_{—}137, 995905 bp, 3.63 cM). In additional examples, the one or more marker locus detected comprises one or more markers within the chromosome interval on linkage group A1 a region of 5 cM, 10 cM, 15 cM, 20 cM, 25 cM, or 30 cM comprising 500405. In still further examples, the one or more marker locus detected comprises one or more markers within the chromosome interval on chromosome 5 (Gm05) flanked by and including nucleotide positions 7677721 and 9097315. In yet further examples, the one or more marker locus detected comprises one or more markers within one or more of the genomic DNA regions of SEQ ID NOs: 1-145. In other examples, the one or more marker locus detected comprises one or more markers within one or more of the genomic regions of SEQ ID NOs: 5, 10, 15, 20, 25, 29, 33, 37, 42, 47, S2, S6, 61, 66, 70, 74, 78, 82, 86, 91, 96, 101, 106, 110, 115, 120, 125, 130, 135, 140, and 145. In some examples, the one or more polymorphism detected may be less than 1 cM, 1 cM, 5 cM, 10 cM, 15 cM, 20 cM, or 30 cM from SEQ ID NO: 1-145.

In some examples, the at least one favorable allele of one or more marker loci is selected from the group consisting of S00405-1-A allele G, Gm05 position 8810680 allele G, S15121-001-Q001 allele T, Gm05 position 8650576 allele T, S15124-001-Q001 allele A, Gm05 position 8671038 allele A, S04776-1-A allele G, Gm05 position 8021614 allele G, S15081-001-Q001 null allele, S05017-1-K1 allele A, S07022-1-K001 allele T, S10456-1-K1 allele A, S15126-001-Q001 allele A, S15071-001-Q001 allele A, S15122-001-Q001 allele G, S13062-1-K1 allele C, S15125-001-Q001 allele T, S15123-001-Q001 allele A, S12985-1-K1 allele A, S13064-1-K1 allele T, S05933-1-K1 allele A, S13078-1-K1 allele G, S13073-1-K1 allele T, S01261-1-A allele A, S14531-001-Q001 allele T, S01282-1-A allele G, S14582-001-Q001 allele C, S10245-1-K1 allele G, S14581-001-Q001 allele T, S10446-001-Q1 allele A, S14561-001-Q001 allele T, S14552-001-Q001 allele G, S14562-001-Q001 allele G, S13012-001-Q002 allele T, and S05107-001-Q002 allele T. In some examples, the SNP haplotype comprises the marker alleles S00405-1-A allele G, S15121-001-Q001 allele T, S15124-001-Q001 allele A, S04776-1-A allele G, S15081-001-Q001 null allele, S05017-1-K1 allele A, S07022-1-K001 allele T, S10456-1-K1 allele A, S15126-001-Q001 allele A, S15071-001-Q001 allele A, S15122-001-Q001 allele G, S13062-1-K1 allele C, S15125-001-Q001 allele T, S15123-001-Q001 allele A, S12985-1-K1 allele A, S13064-1-K1 allele T, S05933-1-K1 allele A, S13078-1-K1 allele G, S13073-1-K1 allele T, S01261-1-A allele A, S14531-001-Q001 allele T, S01282-1-A allele G, S14582-001-Q001 allele C, S10245-1-K1 allele G, S14581-001-Q001 allele T, S10446-001-Q1 allele A, S14561-001-Q001 allele T, S14552-001-Q001 allele G, S14562-001-Q001 allele G, S13012-001-Q002 allele T, and S05107-001-Q002 allele T. In some examples, the SNP haplotype comprises the marker alleles Gm05 position 8810680 allele G, Gm05 position 8650576 allele T, Gm05 position 8671038 allele A, Gm05 position 8021614 allele G, Gm05 position 8712346 null allele, Gm05 position 9097414 allele A, Gm05 position 9002798 allele T, Gm05 position 8796827 allele A, Gm05 position 8809479 allele A, Gm05 position 8659968 allele G, Gm05 position 8622812 allele C, Gm05 position 8673968 allele T, Gm05 position 8660316 allele A, Gm05 position 8659986 allele A, Gm05 position 8173288 allele T, Gm05 position 7943632 allele A, Gm05 position 7850805 allele G, Gm05 position 7677721 allele T, Gm05 position 620718 allele A, Gm05 position 2012649 allele G, Gm05 position 2578312 allele C, Gm05 position 2573680 allele G, Gm05 position 2703606 allele T, Gm05 position 3271804 allele A, Gm05 position 3603395 allele T, Gm05 position 3604317 allele G, Gm05 position 3597393 allele G, Gm05 position 5711938 allele T, and Gm05 position 6852084 allele T. In other examples, the SNP haplotype comprises the marker alleles. In other examples, the haplotype comprises two or more favorable alleles from the set of alleles described in Table 6. In some examples, the haplotype may comprise a combination of favorable and unfavorable alleles.

Detecting may comprise amplifying the marker locus or a portion of the marker locus and detecting the resulting amplified marker amplicon. In particular examples, the amplifying comprises admixing an amplification primer or amplification primer pair and, optionally at least one nucleic acid probe, with a nucleic acid isolated from the first soybean plant or germplasm, wherein the primer or primer pair and optional probe is complementary or partially complementary to at least a portion of the marker locus and is capable of initiating DNA polymerization by a DNA polymerase using the soybean nucleic acid as a template; and, extending the primer or primer pair in a DNA polymerization reaction comprising a DNA polymerase and a template nucleic acid to generate at least one amplicon. In particular examples, the detection comprises real time PCR analysis.

In still further aspects, the information disclosed herein regarding marker alleles and SNP haplotypes can be used to aid in the selection of breeding plants, lines, and populations containing tolerance to iron deficiency, and/or for use in introgression of this trait into elite soybean germplasm, exotic soybean germplasm, or any other soybean germplasm. Also provided is a method for introgressing a soybean QTL, marker, or haplotype associated with iron deficiency tolerance into non-tolerant or less tolerant soybean germplasm. According to the method, markers and/or haplotypes are used to select soybean plants containing the improved tolerance trait. Plants so selected can be used in a soybean breeding program. Through the process of introgression, the QTL, marker, or haplotype associated with improved iron deficiency tolerance is introduced from plants identified using marker-assisted selection (MAS) to other plants. According to the method, agronomically desirable plants and seeds can be produced containing the QTL, marker, or haplotype associated with iron deficiency tolerance from germplasm containing the QTL, marker, or haplotype. Sources of improved tolerance are disclosed below.

Also provided herein is a method for producing a soybean plant adapted for conferring improved iron deficiency tolerance. First, donor soybean plants for a parental line containing the tolerance QTL, marker, and/or haplotype are selected. According to the method, selection can be accomplished via MAS as explained herein. Selected plant material may represent, among others, an inbred line, a hybrid line, a heterogeneous population of soybean plants, or an individual plant. According to techniques well known in the art of plant breeding, this donor parental line is crossed with a second parental line. In some examples, the second parental line is a high yielding line. This cross produces a segregating plant population composed of genetically heterogeneous plants. Plants of the segregating plant population are screened for the tolerance QTL, marker, or haplotype. Further breeding may include, among other techniques, additional crosses with other lines, hybrids, backcrossing, or self-crossing. The result is a line of soybean plants that has improved tolerance to iron deficiency and optionally also has other desirable traits from one or more other soybean lines.

Also provided is a method of soybean plant breeding comprising crossing at least two different soybean parent plants, wherein the parent soybean plants differ in iron deficiency tolerance phenotypic, obtaining a population of progeny soybean seed from said cross, genotyping the progeny soybean seed with at least one genetic marker, and, selecting a subpopulation comprising at least one soybean seed possessing a genotype for improved iron deficiency tolerance, wherein the mean iron deficiency tolerance phenotype of the selected subpopulation is improved as compared to the mean iron deficiency tolerance phenotype of the non-selected progeny. In some examples the mean iron deficiency tolerance phenotype is determined on a scoring scale, for example a scale of 1-9, wherein plants with a score of 1 are completely susceptible and plants with a score of 9 are completely tolerant. In some examples the mean iron deficiency tolerance phenotype of the selected subpopulation of progeny is at least 0.25, 0.5, 0.75, or 1 points greater than the mean iron deficiency tolerance phenotype of the non-selected progeny. In other examples the mean iron deficiency tolerance phenotype of the selected subpopulation of progeny is at least 2, 3, 4, 5, 6, 7, or 8 points greater than the mean iron deficiency tolerance phenotype of the non-selected progeny. In some examples, the two different soybean parent plants also differ by maturity. The maturity groups of the parent plants may differ by one or more maturity subgroups, by one or more maturity groups, or by 1 or more days to maturity. In some examples the parents differ in maturity by at least 10 days, between 10 days-20 days, between 10 days-30 days, by at least 0.1, 0.2, 0.3. 0.4, 0.5, 0.6, 0.7, 0.8, or 0.9 maturity subgroups, by at least 1, 2, 3, 4, 5, 6, 7, 8, 9, 10, 11, or 12 maturity groups. In some examples one parent is adapted for a northern growing region, and the second parent is not adapted for a northern growing region. In some examples the parent adapted for a northern growing region comprises better iron deficiency tolerance than the parent not adapted for a northern growing region. In some examples, the method further comprises obtaining progeny better adapted for a northern growing region.

Soybean plants, seeds, tissue cultures, variants and mutants having improved iron deficiency tolerance produced by the foregoing methods are also provided. Soybean plants, seeds, tissue cultures, variants and mutants comprising one or more of the marker loci, one or more of the favorable alleles, and/or one or more of the haplotypes and having improved iron deficiency tolerance are provided. Also provided are isolated nucleic acids, kits, and systems useful for the identification and/or selection methods disclosed herein.

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference for the purpose cited to the same extent as if each was specifically and individually indicated to be incorporated by reference herein.

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Additionally, as used herein, “comprising” is to be interpreted as specifying the presence of the stated features, integers, steps, or components as referred to, but does not preclude the presence or addition of one or more features, integers, steps, or components, or groups thereof. Thus, for example, a kit comprising one pair of oligonucleotide primers may have two or more pairs of oligonucleotide primers. Additionally, the term “comprising” is intended to include examples encompassed by the terms “consisting essentially of” and “consisting of.” Similarly, the term “consisting essentially of” is intended to include examples encompassed by the term “consisting of.”

Certain definitions used in the specification and claims are provided below. In order to provide a clear and consistent understanding of the specification and claims, including the scope to be given such terms, the following definitions are provided:

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant.

The term “amplifying” in the context of nucleic acid amplification is any process whereby additional copies of a selected nucleic acid (or a transcribed form thereof) are produced. An “amplicon” is an amplified nucleic acid, e.g., a nucleic acid that is produced by amplifying a template nucleic acid by any available amplification method.

“Backcrossing” is a process in which a breeder crosses a progeny variety back to one of the parental genotypes one or more times.

The term “chromosome segment” designates a contiguous linear span of genomic DNA that resides in planta on a single chromosome. “Chromosome interval” refers to a chromosome segment defined by specific flanking marker loci.

“Cultivar” and “variety” are used synonymously and mean a group of plants within a species (e.g., Glycine max) that share certain genetic traits that separate them from other possible varieties within that species. Soybean cultivars are inbred lines produced after several generations of self-pollinations. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic association or linkage relationships among loci on one or more chromosomes (or linkage groups) within a given species, generally depicted in a diagrammatic or tabular form.

“Genotype” is a description of the allelic state at one or more loci in a genome.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Introgression” means the entry or introduction of a gene, QTL, marker, haplotype, marker profile, trait, or trait locus from the genome of one plant into the genome of another plant.

The terms “label” and “detectable label” refer to a molecule capable of detection. A detectable label can also include a combination of a reporter and a quencher, such as are employed in FRET probes or TAQMAN® probes. The term “reporter” refers to a substance or a portion thereof that is capable of exhibiting a detectable signal, which signal can be suppressed by a quencher. The detectable signal of the reporter is, e.g., fluorescence in the detectable range. The term “quencher” refers to a substance or portion thereof that is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a subline has been derived by inbreeding the seed from an individual soybean plant selected at the F3 to F5 generation until the residual segregating loci are “fixed” or homozygous across most or all loci. Commercial soybean varieties (or lines) are typically produced by aggregating (“bulking”) the self-pollinated progeny of a single F3 to F5 plant from a controlled cross between two genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to the tendency for alleles tend to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers, the lower the frequency of recombination, the greater the degree of linkage.

“Linkage disequilibrium” is a non-random association of alleles at two or more loci and can occur between unlinked markers. It is based on allele frequencies within a population and is influenced by but not dependent on linkage. Linkage disequilibrium is typically detected when alleles segregate from parents to offspring with a greater frequency than expected from their individual frequencies.

“Linkage group” refers to traits or markers that co-segregate. A linkage group generally corresponds to a chromosomal region containing genetic material that encodes the traits or markers.

“Locus” is a defined segment of DNA.

A “map location,” a “map position,” or a “relative map position” is an assigned location on a genetic map relative to linked genetic markers where a specified marker can be found within a given species. Map positions are generally provided in centimorgans (cM), unless otherwise indicated, genetic positions provided are based on the Glycine max consensus map v 4.0 as provided by Hyten et al. (2010) Crop Sci 50:960-968. A “physical position” or “physical location” is the position, typically in nucleotide bases, of a particular nucleotide, such as a SNP nucleotide, on the chromosome. Unless otherwise indicated, the physical position within the soybean genome provided is based on the Glyma 1.0 genome sequence described in Schmutz et al. (2010) Nature 463:178-183, available from the Phytozome website (phytozome-dot-net/soybean).

“Mapping” is the process of defining the association and relationships of loci through the use of genetic markers, populations segregating for the markers, and standard genetic principles of recombination frequency.

“Marker” or “molecular marker” is a term used to denote a nucleic acid or amino acid sequence that is sufficiently unique to characterize a specific locus on the genome. Any detectible polymorphic trait can be used as a marker so long as it is inherited differentially and exhibits linkage disequilibrium with a phenotypic trait of interest.

“Marker assisted selection” refers to the process of selecting a desired trait or traits in a plant or plants by detecting one or more nucleic acids from the plant, where the nucleic acid is associated with or linked to the desired trait, and then selecting the plant or germplasm possessing those one or more nucleic acids.

“Maturity Group” is an agreed-on industry division of groups of varieties, based on the zones in which they are adapted primarily according to day length and/or latitude. Soybean varieties are grouped into 13 maturity groups, depending on the climate and latitude for which they are adapted. Soybean maturities are divided into relative maturity groups (denoted as 000, 00, 0, I, II, III, IV, V, VI, VII, VIII, IX, X, or 000, 00, 0, 1, 2, 3, 4, 5, 6, 7, 8, 9, 10). These maturity groups are given numbers, with numbers 000, 00, 0 and 1 typically being adapted to Canada and the northern United States, groups VII, VIII and IX being grown in the southern regions, and Group X is tropical. Within a maturity group are sub-groups. A sub-group is a tenth of a relative maturity group (for example 1.3 would indicate a group 1 and subgroup 3). Within narrow comparisons, the difference of a tenth of a relative maturity group equates very roughly to a day difference in maturity at harvest.

“Haplotype” refers to a combination of particular alleles present within a particular plant's genome at two or more linked marker loci, for instance at two or more loci on a particular linkage group. For instance, in one example, two specific marker loci on LG A1 are used to define a haplotype for a particular plant. In still further examples, 3, 4, 5, 6, 7, 8, 9, 10, 11, 12, 13, 14, 15, 16, 17, 18, 19, 20, or more linked marker loci are used to define a haplotype for a particular plant.

As used herein, a “marker profile” means a combination of particular alleles present within a particular plant's genome at two or more marker loci which are not linked, for instance two or more loci on two or more different linkage groups or two or more chromosomes. For instance, in one example, one marker locus on LG A1 and a marker locus on another linkage group are used to define a marker profile for a particular plant. In certain other examples a plant's marker profile comprises one or more haplotypes. In some examples, the marker profile further includes at least one marker locus on LG A1 associated with iron deficiency tolerance. In some examples, the marker profile encompasses two or more loci for the same trait, such as iron deficiency tolerance. In other examples, the marker profile encompasses two or more loci associated with two or more traits of interest, such as iron deficiency tolerance and a second trait of interest.

The term “plant” includes reference to an immature or mature whole plant, including a plant from which seed or grain or anthers have been removed. Seed or embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells, and the like.

“Polymorphism” means a change or difference between two related nucleic acids. A “nucleotide polymorphism” refers to a nucleotide that is different in one sequence when compared to a related sequence when the two nucleic acids are aligned for maximal correspondence.

“Polynucleotide,” “polynucleotide sequence,” “nucleic acid sequence,” “nucleic acid fragment,” and “oligonucleotide” are used interchangeably herein to indicate a polymer of nucleotides that is single- or multi-stranded, that optionally contains synthetic, non-natural, or altered RNA or DNA nucleotide bases. A DNA polynucleotide may be comprised of one or more strands of cDNA, genomic DNA, synthetic DNA, or mixtures thereof.

“Primer” refers to an oligonucleotide which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

“Probe” refers to an oligonucleotide that is complementary (though not necessarily fully complementary) to a polynucleotide of interest and forms a duplexed structure by hybridization with at least one strand of the polynucleotide of interest. Typically, probes are oligonucleotides from 10 to 50 nucleotides in length, but longer or shorter sequences can be employed. A probe can further contain a detectable label.

“Quantitative trait loci” or “QTL” refer to the genetic elements controlling a quantitative trait.

“Recombination frequency” is the frequency of a crossing over event (recombination) between two genetic loci. Recombination frequency can be observed by following the segregation of markers and/or traits during meiosis.

“Tolerance and “improved tolerance” are used interchangeably herein and refer to any type of increase in resistance or tolerance to, or any type of decrease in susceptibility. A “tolerant plant” or “tolerant plant variety” need not possess absolute or complete tolerance. Instead, a “tolerant plant,” “tolerant plant variety,” or a plant or plant variety with “improved tolerance” will have a level of resistance or tolerance which is higher than that of a comparable susceptible plant or variety.

“Self crossing” or “self pollination” or “selfing” is a process through which a breeder crosses a plant with itself; for example, a second-generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome.

The term “yield” refers to the productivity per unit area of a particular plant product of commercial value. For example, yield of soybean is commonly measured in bushels of seed per acre or metric tons of seed per hectare per season. Yield is affected by both genetic and environmental factors.

An “isolated” or “purified” polynucleotide or polypeptide, or biologically active portion thereof, is substantially or essentially free from components that normally accompany or interact with the polynucleotide or polypeptide as found in its naturally occurring environment. Typically, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein, culture media, or other chemical components.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Sambrook”).

Iron deficiency severely limits growth of soybeans in several regions of North America, particularly in poorly drained calcareous (heavy lime) soils in parts of Minnesota, the Dakotas, Nebraska and Iowa. Iron deficiency chlorosis is a complex plant disorder often associated with high pH soils and soils containing soluble salts where chemical conditions reduce the availability of iron. Environmental and soil conditions including compaction, excessive soil moisture and low soil temperatures can contribute to iron chlorosis severity, which can be differentially impact different areas of fields.

Iron is found in soil mainly as insoluble oxyhydroxide polymers (FeOOH) that are extremely insoluble (10⁻¹⁷ M) at neutral pH. Since the optimal concentration of soluble Fe for plant growth is approximately 10⁻⁶ M, plants have at least two different strategies to access the iron they need from soil (Fox & Guerinot (1998) Ann Rev Plant Physiol Plant Mol Biol 49:669-96). Strategy I is used by all plants except grasses (Marschner et al. (1986) J Plant Nutr 9:3-7). This strategy involves a multi-step process, beginning with the plants releasing H+ ions into the soil from the roots via proton pump activity from an H+ATPase, which lowers soil pH. The lowered pH leads to the dissociation of Fe(OH), complexes into ferrous ions. Fe(III) is reduced to the more soluble Fe(II) by a membrane-bound ferric chelate reductase located in root epidermal cells. Following reduction, a separate transport protein moves the reduced iron across the root plasma membrane. A gene IRT1 (iron regulated transporter) which codes for the transport protein has also been found in Arabidopsis (Eide et al. (1996) PNAS 93:5624-5628). This same transport protein has been shown to transport manganese, zinc, and cobalt as well (Korshunova et al. (1999) Plant Mol. Biol 40:37-44).

High carbonate levels in the soil are the main source of iron deficiency chlorosis in soybean. Other stresses, such as cold temperature, SCN infection, water saturated soils, or herbicide application may increase chlorosis. Bicarbonates can also impede the movement of iron to young leaves once it is absorbed by the roots (Barker & Pilbeam (ed.) 2007. Handbook of Plant Nutrition Vol. 117 ed. 1:335-337. Taylor & Francis Publ., New York, Philadelphia, Oxford, Melbourne, Stockholm, Beijing, New Delhi, Johannesburg, Singapore and Tokyo). Iron deficiency symptoms range from slight yellowing of leaves to stunting, severe chlorosis, and sometimes death of plants in affected fields.

While iron availability can be modulated environmentally to some extent (e.g., by modifying soil pH or adding soluble iron, applying foliar iron treatments, or applying iron to seed), these approaches can cause unwanted side effects in the soybean or the environment and also add to soybean production costs. Some treatments, such as iron treatment of seed, display inconsistent results in different cultivars or field environments. Despite these difficulties, most producers currently rely on the use of seed, foliar, or soil treatments to reduce iron deficiency chlorosis (see, e.g., Weirsma (2002) Cropping Issues in Northwest Minnesota 1(7):1-2); Goos & Germain (2001) Communications in Soil Science and Plant Analysis 32:2317-2323).

For some time, soybean producers have sought to develop iron deficiency tolerant plants as a cost-effective alternative or supplement to standard foliar, soil and/or seed treatments (e.g., Hintz et al. (1987) Crop Sci 28:369-370). Other studies also suggest that cultivar selection is more reliable and universally applicable than foliar sprays or iron seed treatment methods, though environmental and cultivar selection methods can also be used effectively in combination. See also, Goos & Johnson (2000) Agron J 92:1135-1139; and Goos & Johnson (2001) J Plant Nutr 24:1255-1268.

The advent of molecular genetic markers has facilitated mapping and selection of agriculturally important traits in soybean. Markers tightly linked to tolerance genes are an asset in the rapid identification of tolerant soybean lines on the basis of genotype by the use of marker assisted selection (MAS). Introgressing tolerance genes into a desired cultivar would also be facilitated by using suitable DNA markers.

Soybean cultivar improvement for iron deficiency tolerance can be performed using classical breeding methods, or, by using marker assisted selection (MAS). Genetic markers for iron deficiency tolerance/susceptibility have been identified (e.g., Lin et al. (2000) J Plant Nutr 23:1929-1939; Diers et al. (1992) J Plant Nutr 15:2127-2136; Lin et al. (1997) Mol Breed 3:219-229; Charlson et al. (2003) J Plant Nutr 26:2267-2276; Charlson et al. (2005) Crop Sci 45:2394-2399). Studies suggest that marker assisted selection is particularly beneficial when selecting plants for iron deficiency tolerance (e.g., Charlson et al. (2003) J Plant Nutr 26:2267-2276).

Provided are markers, haplotypes, and/or marker profiles associated with tolerance of soybean plants to iron deficiency, as well as related primers and/or probes and methods for the use of any of the foregoing for identifying and/or selecting soybean plants with improved tolerance to iron deficiency. A method for determining the presence or absence of at least one allele of a particular marker or haplotype associated with tolerance to iron deficiency comprises analyzing genomic DNA from a soybean plant or germplasm to determine if at least one, or a plurality, of such markers is present or absent and if present, determining the allelic form of the marker(s). If a plurality of markers on a single linkage group are investigated, this information regarding the markers present in the particular plant or germplasm can be used to determine a haplotype for that plant/germplasm.

In certain examples, plants or germplasm are identified that have at least one favorable allele, marker, and/or haplotype that positively correlate with tolerance or improved tolerance. However, in other examples, it is useful to identify alleles, markers, and/or haplotypes that negatively correlate with tolerance, for example to eliminate such plants or germplasm from subsequent rounds of breeding. Plants or germplasm having tolerance or improved tolerance to iron deficiency chlorosis are provided.

Any marker associated with an iron deficiency tolerance QTL is useful. Further, any suitable type of marker can be used, including Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). Additionally, other types of molecular markers known in the art or phenotypic traits may also be used as markers in the methods.

Markers that map closer to an iron deficiency tolerance QTL are generally used over markers that map farther from such a QTL. Marker loci are especially useful when they are closely linked to an iron deficiency tolerance QTL. Thus, in one example, marker loci display an inter-locus cross-over frequency of about 10% or less, about 9% or less, about 8% or less, about 7% or less, about 6% or less, about 5% or less, about 4% or less, about 3% or less, about 2% or less, about 1% or less, about 0.75% or less, about 0.5% or less, or about 0.25% or less with an iron deficiency tolerance QTL to which they are linked. Thus, the loci are separated from the QTL to which they are linked by about 10 cM, 9 cM, 8 cM, 7 cM, 6 cM, 5 cM, 4 cM, 3 cM, 2 cM, 1 cM, 0.75 cM, 0.5 cM, or 0.25 cM or less.

In certain examples, multiple marker loci that collectively make up a haplotype and/or a marker profile are investigated, for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, or more marker loci.

In addition to the markers discussed herein, information regarding useful soybean markers can be found, for example, on the USDA's Soybase website, available at www.soybase.org. A number of soybean markers have been mapped and linkage groups created, as described in Cregan et al. (1999) Crop Sci 39:1464-90, Choi et al. (2007) Genetics 176:685-96, and Hyten, et al. (2010) Crop Sci 50:960-968, each of which is herein incorporated by reference in its entirety, including any supplemental materials associated with the publication. Many soybean markers are publicly available at the USDA affiliated soybase website (at soybase-dot-org). One of skill in the art will recognize that the identification of favorable marker alleles may be germplasm-specific. One of skill will also recognize that methods for identifying the favorable alleles are routine and well known in the art, and furthermore, that the identification and use of such favorable alleles is well within the scope of the invention.

The use of marker assisted selection (MAS) to select a soybean plant or germplasm based upon detection of a particular marker or haplotype of interest is provided. For instance, in certain examples, a soybean plant or germplasm possessing a certain predetermined favorable marker allele or haplotype will be selected via MAS. Using MAS, soybean plants or germplasm can be selected for markers or marker alleles that positively correlate with tolerance, without actually raising soybean and measuring for tolerance (or, contrawise, soybean plants can be selected against if they possess markers that negatively correlate with tolerance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some examples, molecular markers are detected using a suitable amplification-based detection method. Typical amplification methods include various polymerase based replication methods, including the polymerase chain reaction (PCR), ligase mediated methods, such as the ligase chain reaction (LCR), and RNA polymerase based amplification (e.g., by transcription) methods. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage & Caruthers (1981) Tetrahedron Letts 22:1859-1862, e.g., using a commercially available automated synthesizer, e.g., as described in Needham-VanDevanter et al. (1984) Nucl Acids Res 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair, or probe. For example, primers can be designed using any suitable software program, such as LASERGENE® or Primer3.

The primers are not limited to generating an amplicon of any particular size. For example, the primers used to amplify the marker loci and alleles herein are not limited to amplifying the entire region of the relevant locus. In some examples, marker amplification produces an amplicon at least 20 nucleotides in length, or alternatively, at least 50 nucleotides in length, or alternatively, at least 100 nucleotides in length, or alternatively, at least 200 nucleotides in length, or alternatively, at least 300 nucleotides in length, or alternatively, at least 400 nucleotides in length, or alternatively, at least 500 nucleotides in length, or alternatively, at least 1000 nucleotides in length, or alternatively, at least 2000 nucleotides in length or more.

PCR, RT-PCR, and LCR are common amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous references, such as Mullis et al. (1987) U.S. Pat. No. 4,683,202; Arnheim & Levinson (1990) C&EN 36-47; Kwoh et al. (1989) Proc Natl Acad Sci USA 86:1173; Guatelli et al. (1990) Proc Natl Acad Sci USA 87:1874; Lomell et al. (1989) J Clin Chem 35:1826; Landegren et al. (1988) Science 241:1077-1080; Van Brunt (1990) Biotechnology 8:291-294; Wu & Wallace (1989) Gene 4:560; Barringer et al. (1990) Gene 89:117; and Sooknanan & Malek (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype alleles, such as SNP alleles, are provided. For example, exemplary primers and probes are provided in Table 2. However, one of skill will immediately recognize that other primer and probe sequences could also be used. For instance, primers to either side of the given primers can be used in place of the given primers, so long as the primers can amplify a region that includes the allele to be detected, as can primers and probes directed to other marker loci. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Labeling strategies for labeling nucleic acids and their corresponding detection strategies can be found, e.g., in Haugland (1996) Handbook of Fluorescent Probes and Research Chemicals Sixth Edition by Molecular Probes, Inc. (Eugene, Oreg.); or Haugland (2001) Handbook of Fluorescent Probes and Research Chemicals Eighth Edition by Molecular Probes, Inc. (Eugene, Oreg.).

Detectable labels may also include reporter-quencher pairs, such as are employed in Molecular Beacon and TAQMAN® probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland (2001) Handbook of Fluorescent Probes and Research Chemicals Eighth Edition by Molecular Probes, Inc. (Eugene, Oreg.), the content of which is incorporated herein by reference.

In certain examples, reporter-quencher pairs are selected from xanthene dyes including fluorescein and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters is the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from available from Applied Biosystems, and the like. Suitable examples of quenchers may be selected from 6-carboxy-tetramethyl-rhodamine, 4-(4-dimethylaminophenylazo)benzoic acid (DABYL), tetramethylrhodamine (TAMRA), BHQ-0™, BHQ-1™, BHQ-2™, and BHQ-3™, each of which are available from Biosearch Technologies, Inc. of Novato, Calif., QSY-7™, QSY-9™, QSY-21™ and QSY-35™, each of which are available from Molecular Probes, Inc., and the like.

In one aspect, real time PCR or LCR is performed on the amplification mixtures described herein, e.g., using molecular beacons or TAQMAN® probes. A molecular beacon (MB) is an oligonucleotide that, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone et al. (1995) Nucl Acids Res 26:2150-2155; Tyagi & Kramer (1996) Nat Biotechnol 14:303-308; Blok & Kramer (1997) Mol Cell Probes 11:187-194; Hsuih et al. (1997) J Clin Microbiol 34:501-507; Kostrikis et al. (1998) Science 279:1228-1229; Sokol et al. (1998) Proc Natl Acad Sci USA 95:11538-11543; Tyagi et al. (1998) Nat Biotechnol 16:49-53; Bonnet et al. (1999) Proc Natl Acad Sci USA 96:6171-6176; Fang et al. (1999) J Am Chem Soc 121:2921-2922; Marras et al. (1999) Genet Anal Biomol Eng 14:151-156; and, Vet et al. (1999) Proc Natl Acad Sci USA 96:6394-6399. Additional details regarding MB construction and use are also found in the patent literature, e.g., U.S. Pat. Nos. 5,925,517; 6,150,097; and 6,037,130.

Another real-time detection method is the 5′-exonuclease detection method, also called the TAQMAN® assay, as set forth in U.S. Pat. Nos. 5,804,375; 5,538,848; 5,487,972; and 5,210,015, each of which is hereby incorporated by reference in its entirety. In the TAQMAN® assay, a modified probe, typically 10-30 nucleotides in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are typically attached to the probe within a few nucleotides of one another, usually within 30 nucleotides of one another, or within 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

One example of a suitable real-time detection technique that does not use a separate probe that binds intermediate to the two primers is the KASPar detection system/method, which is well known in the art. In KASPar, two allele specific primers are designed such that the 3′ nucleotide of each primer hybridizes to the polymorphic base. For example, if the SNP is an A/C polymorphism, one of the primers would have an “A” in the 3′ position, while the other primer would have a “C” in the 3′ position. Each of these two allele specific primers also has a unique tail sequence on the 5′ end of the primer. A common reverse primer is employed that amplifies in conjunction with either of the two allele specific primers. Two 5′ fluor-labeled reporter oligos are also included in the reaction mix, one designed to interact with each of the unique tail sequences of the allele-specific primers. Lastly, one quencher oligo is included for each of the two reporter oligos, the quencher oligo being complementary to the reporter oligo and being able to quench the fluor signal when bound to the reporter oligo. During PCR, the allele-specific primers and reverse primers bind to complementary DNA, allowing amplification of the amplicon to take place. During a subsequent cycle, a complementary nucleic acid strand containing a sequence complementary to the unique tail sequence of the allele-specific primer is created. In a further cycle, the reporter oligo interacts with this complementary tail sequence, acting as a labeled primer. Thus, the product created from this cycle of PCR is a fluorescently-labeled nucleic acid strand. Because the label incorporated into this amplification product is specific to the allele specific primer that resulted in the amplification, detecting the specific fluor presenting a signal can be used to determine the SNP allele that was present in the sample.

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook et al. Molecular Cloning—A Laboratory Manual (3d ed.) Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”); and, PCR Protocols A Guide to Methods and Applications (Innis et al., eds) Academic Press Inc. San Diego, Calif. (1990) (“Innis”). Additional details regarding detection of nucleic acids in plants can also be found, e.g., in Plant Molecular Biology (1993) Croy (ed.) BIOS Scientific Publishers, Inc.

Other techniques for detecting SNPs can also be employed, such as allele specific hybridization (ASH) or nucleic acid sequencing techniques. ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Isolated polynucleotide or fragments thereof are capable of specifically hybridizing to other nucleic acid molecules under appropriate conditions. In one example, the nucleic acid molecules contain any of SEQ ID NOs: 1-145, complements thereof and fragments thereof. In another aspect, the nucleic acid molecules of the present invention include nucleic acid molecules that hybridize, for example, under high or low stringency, substantially homologous sequences, or that have both to these molecules. Conventional stringency conditions are described by Sambrook et al. In: Molecular Cloning, A Laboratory Manual, 2nd Edition, Cold Spring Harbor Press, Cold Spring Harbor, N.Y. (1989)), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed. Appropriate stringency conditions that promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In some examples, an a marker locus will specifically hybridize to one or more of the nucleic acid molecules set forth in SEQ ID NOs: 1-145 or complements thereof or fragments of either under moderately stringent conditions, for example at about 2.0×SSC and about 65° C. In an aspect, a nucleic acid of the present invention will specifically hybridize to one or more SEQ ID NOs: 1-145 or complements or fragments of either under high stringency conditions.

In some examples, a marker associated with iron deficiency tolerance comprises any one of SEQ ID NOs: 1-145 or complements or fragments thereof. In other examples, a marker has between 80%, 85%, 90%, 91%, 92%, 93%, 94%, 95%, 96%, 97%, 98%, 99%, or 100% sequence identity to any one of SEQ ID NOs: 1-145 or complements or fragments thereof. Unless otherwise stated, percent sequence identity is determined using the GAP program is default parameters for nucleic acid alignment (Accelrys, San Diego, Calif., USA).

Traits or markers are considered herein to be linked if they generally co-segregate. A 1/100 probability of recombination per generation is defined as a map distance of 1.0 centiMorgan (1.0 cM). The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosome segment are also genetically linked, typically within a genetic recombination distance of less than or equal to 50 centimorgans (cM), e.g., about 49, 40, 30, 20, 10, 9, 8, 7, 6, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 9%, 8%, 7%, 6%, 5%, 4%, 3%, 2%, 1%, 0.75%, 0.5%, or 0.25% or less. Closely linked markers display a cross over frequency with a given marker of about 10% or less (the given marker is within about 10 cM of a closely linked marker). Put another way, closely linked loci co-segregate at least about 90% of the time. With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less.

When referring to the relationship between two genetic elements, such as a genetic element contributing to tolerance and a proximal marker, “coupling” phase linkage indicates the state where the “favorable” allele at the tolerance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for tolerance) is physically linked with an “unfavorable” allele at the proximal marker locus, and the two “favorable” alleles are not inherited together (i.e., the two loci are “out of phase” with each other).

Markers are used to define a specific locus on the soybean genome. Each marker is therefore an indicator of a specific segment of DNA, having a unique nucleotide sequence. Map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed. Molecular markers have been widely used to determine genetic composition in soybeans.

Favorable genotypes associated with at least trait of interest may be identified by one or more methodologies. In some examples one or more markers are used, including but not limited to AFLPs, RFLPs, ASH, SSRs, SNPs, indels, padlock probes, molecular inversion probes, microarrays, sequencing, and the like. In some methods, a target nucleic acid is amplified prior to hybridization with a probe. In other cases, the target nucleic acid is not amplified prior to hybridization, such as methods using molecular inversion probes (see, for example Hardenbol et al. (2003) Nat Biotech 21:673-678). In some examples, the genotype related to a specific trait is monitored, while in other examples, a genome-wide evaluation including but not limited to one or more of marker panels, library screens, association studies, microarrays, gene chips, expression studies, or sequencing such as whole-genome resequencing and genotyping-by-sequencing (GBS) may be used. In some examples, no target-specific probe is needed, for example by using sequencing technologies, including but not limited to next-generation sequencing methods (see, for example, Metzker (2010) Nat Rev Genet 11:31-46; and, Egan et al. (2012) Am J Bot 99:175-185) such as sequencing by synthesis (e.g., Roche 454 pyrosequencing, Illumina Genome Analyzer, and Ion Torrent PGM or Proton systems), sequencing by ligation (e.g., SOLiD from Applied Biosystems, and Polnator system from Azco Biotech), and single molecule sequencing (SMS or third-generation sequencing) which eliminate template amplification (e.g., Helicos system, and PacBio RS system from Pacific BioSciences). Further technologies include optical sequencing systems (e.g., Starlight from Life Technologies), and nanopore sequencing (e.g., GridION from Oxford Nanopore Technologies). Each of these may be coupled with one or more enrichment strategies for organellar or nuclear genomes in order to reduce the complexity of the genome under investigation via PCR, hybridization, restriction enzyme (see, e.g., Elshire et al. (2011) PLoS ONE 6:e19379), and expression methods. In some examples, no reference genome sequence is needed in order to complete the analysis.

In some examples, markers within 1 cM, 5 cM, 10 cM, 15 cM, or 30 cM of SEQ ID NO: 17-24 are provided. Similarly, one or more markers mapped within 1, 5, 10, 20 and 30 cM or less from the markers provided can be used for the selection or introgression of the region associated with iron deficiency tolerance. In other examples, any marker that is linked with SEQ ID NOs: 1-145 and associated with iron deficiency is provided. In other examples, markers provided include a substantially a nucleic acid molecule within 5 kb, 10 kb, 20 kb, 30 kb, 100 kb, 500 kb, 1,000 kb, 10,000 kb, 25,000 kb, or 50,000 kb of a marker selected from the group consisting of SEQ ID NOs: 1-145.

Real-time amplification assays, including MB or TAQMAN® based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 13-18 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Introgression of iron deficiency tolerance into less tolerant soybean germplasm is provided. Any method for introgressing a QTL or marker into soybean plants known to one of skill in the art can be used. Typically, a first soybean germplasm that contains tolerance to iron deficiency derived from a particular marker or haplotype and a second soybean germplasm that lacks such tolerance derived from the marker or haplotype are provided. The first soybean germplasm may be crossed with the second soybean germplasm to provide progeny soybean germplasm. These progeny germplasm are screened to determine the presence of iron deficiency tolerance derived from the marker or haplotype, and progeny that tests positive for the presence of tolerance derived from the marker or haplotype are selected as being soybean germplasm into which the marker or haplotype has been introgressed. Methods for performing such screening are well known in the art and any suitable method can be used.

One application of MAS is to use the tolerance markers or haplotypes to increase the efficiency of an introgression or backcrossing effort aimed at introducing a tolerance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (tolerance, along with any other available markers for yield, disease tolerance, etc.). Any of the disclosed marker alleles or haplotypes can be introduced into a soybean line via introgression, by traditional breeding (or introduced via transformation, or both) to yield a soybean plant with superior agronomic performance. The number of alleles associated with tolerance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with tolerance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant that comprises at least one of the markers or haplotypes associated with tolerance, such that the progeny are capable of inheriting the marker or haplotype.

Often, a method is applied to at least one related soybean plant such as from progenitor or descendant lines in the subject soybean plants pedigree such that inheritance of the desired tolerance can be traced. The number of generations separating the soybean plants being subject to the methods will generally be from 1 to 20, commonly 1 to 5, and typically 1, 2, or 3 generations of separation, and quite often a direct descendant or parent of the soybean plant will be subject to the method (i.e., 1 generation of separation).

Genetic diversity is important for long-term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool.

For example, the markers, haplotypes, primers, and probes can be used for MAS involving crosses of elite lines to exotic soybean lines (elite×exotic) by subjecting the segregating progeny to MAS to maintain major yield alleles, along with the tolerance marker alleles herein.

As an alternative to standard breeding methods of introducing traits of interest into soybean (e.g., introgression), transgenic approaches can also be used to create transgenic plants with the desired traits. In these methods, exogenous nucleic acids that encode a desired QTL, marker, or haplotype are introduced into target plants or germplasm. For example, a nucleic acid that codes for an iron deficiency tolerance trait is cloned, e.g., via positional cloning, and introduced into a target plant or germplasm.

Experienced plant breeders can recognize iron deficiency tolerant soybean plants in the field, and can select the tolerant individuals or populations for breeding purposes or for propagation. In this context, the plant breeder recognizes “tolerant” and “non-tolerant” or “susceptible” soybean plants. However, plant tolerance is a phenotypic spectrum consisting of extremes in tolerance and susceptibility, as well as a continuum of intermediate tolerance phenotypes. Evaluation of these intermediate phenotypes using reproducible assays are of value to scientists who seek to identify genetic loci that impart tolerance, to conduct marker assisted selection for tolerant populations, and to use introgression techniques to breed a tolerance trait into an elite soybean line, for example.

To that end, screening and selection of tolerant soybean plants may be performed, for example, by exposing plants to iron deficiency in fields or field areas which have produced iron deficiency chlorosis symptoms in soybean consistently in past years, and selecting those plants showing tolerance to iron deficiency. An exemplary iron deficiency chlorosis scoring system is shown in the Examples (Example 1), but any other scoring system known in the art may be used (see, e.g., Wang et al. (2008) Theor Appl Genet 116:777-787).

In some examples, a kit for detecting markers or haplotypes, and/or for correlating the markers or haplotypes with a desired phenotype (e.g., iron deficiency tolerance), are provided. Thus, a typical kit can include a set of marker probes and/or primers configured to detect at least one favorable allele of one or more marker locus associated with tolerance, improved tolerance, or susceptibility to iron deficiency. These probes or primers can be configured, for example, to detect the marker alleles noted in the tables and examples herein, e.g., using any available allele detection format, such as solid or liquid phase array based detection, microfluidic-based sample detection, etc. The kits can further include packaging materials for packaging the probes, primers, or instructions; controls, such as control amplification reactions that include probes, primers, and/or template nucleic acids for amplifications; molecular size markers; or the like.

System or kit instructions that describe how to use the system or kit and/or that correlate the presence or absence of the allele with the predicted tolerance or susceptibility phenotype are also provided. For example, the instructions can include at least one look-up table that includes a correlation between the presence or absence of the favorable allele(s) and the predicted tolerance or improved tolerance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector.

Isolated nucleic acids comprising a nucleic acid sequence coding for tolerance or susceptibility to iron deficiency, or capable of detecting such a phenotypic trait, or sequences complementary thereto, are also included. In certain examples, the isolated nucleic acids are capable of hybridizing under stringent conditions to nucleic acids of a soybean cultivar phenotyped for iron deficiency tolerance, to detect loci associated with iron deficiency tolerance, including one or more of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107. In some examples the isolated nucleic acids are markers, for example markers selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, S14581-001-Q001, S10446-001-Q1, S14561-001-Q001, S14552-001-Q001, S14562-001-Q001, S13012-001-Q002, and S05107-001-Q002. In some examples the nucleic acid is one of more polynucleotides selected from the group consisting of SEQ ID NOs: 1-145. In some examples the nucleic acid is one of more polynucleotides selected from the group consisting of SEQ ID NOs: 5, 10, 15, 20, 25, 29, 33, 37, 42, 47, 52, 56, 61, 66, 70, 74, 78, 82, 86, 91, 96, 101, 106, 110, 115, 120, 125, 130, 135, 140, and 145. Vectors comprising one or more of such nucleic acids, expression products of such vectors expressed in a host compatible therewith, antibodies to the expression product (both polyclonal and monoclonal), and antisense nucleic acids are also included. In some examples, one or more of these nucleic acids is provided in a kit.

As the parental line having iron deficiency tolerance, any line known to the art or disclosed herein may be used. Also included are soybean plants produced by any of the foregoing methods. Seed of a soybean germplasm produced by crossing a soybean variety having a marker or haplotype associated with iron deficiency tolerance with a soybean variety lacking such marker or haplotype, and progeny thereof, is also included.

The present invention is illustrated by the following examples. The foregoing and following description of the present invention and the various examples are not intended to be limiting of the invention but rather are illustrative thereof. Hence, it will be understood that the invention is not limited to the specific details of these examples.

### EXAMPLES

### Example 1

A mapping population comprising 460 individual plants from a F2 mapping populations derived by crossing the iron deficiency tolerant line 90M02 with iron deficiency susceptible lines 92M01 was generated. The population was visually scored for symptoms of iron deficiency chlorosis in late June to mid-July 2011 at the V3 stage (three nodes starting with the first unifoliate leaves). The visual evaluation criteria and scoring scale are shown in Table 1. Phenotypic scores were generated for 257 of the genotyped progeny tested at three locations and reported as the best linear unbiased prediction (BLUP) score. The phenotypic datasets showed normal distributions across the score space.

TABLE 1

| Score | Symptoms |
|---|---|
| 9 | All plants are normal green color |
| 8 | A few plants are showing very light chlorosis on 1 or 2 leaves |
| 7 | <50% of the plants show mild chlorosis (light green leaves) |
| 6 | >50% of the plants show mild chlorosis, but no necrosis seen |
| | on leaves |
| 5 | Most plants are light green to yellow, no necrosis seen on leaves. |
| | Most plants are stunted ~50-75% of normal height |
| 4 | Most plants are yellow, necrosis seen on edges of less than half |
| | the leaves. |
| | Most plants are ~50% of normal height |
| 3 | Most plants are yellow, necrosis seen on most leaves. Most plants |
| | are ~20-40% of normal height |
| 2 | Most leaves are almost dead, most stems are still green. Plants |
| | are severely stunted ~10-20% of normal height |
| 1 | Most plants are completely dead. Live plants are ~10% of normal |
| | height, and have very little living tissue |

Genomic DNA was extracted from leaf tissue of each progeny using a modification of the CTAB (cetyltriethylammonium bromide, Sigma H5882) method described by Stacey & Isaac (Methods in Molecular Biology, Vol. 28: Protocols for Nucleic Acid Analysis by Nonradioactive Probes, Ed: Isaac, Humana Press Inc., Totowa, N.J. 1994, Ch 2, pp. 9-15). Approximately 100-200 mg of tissue was ground into powder in liquid nitrogen and homogenised in 1 ml of CTAB extraction buffer (2% CTAB, 0.02 M EDTA, 0.1 M Tris-Cl pH 8, 1.4 M NaCl, 25 mM DTT) for 30 min at 65° C. Homogenised samples were cooled at room temperature for 15 min before a single protein extraction with approximately 1 ml 24:1 v/v chloroform:octanol was done. Samples were centrifuged for 7 min at 13,000 rpm and the upper layer of supernatant was collected using wide-mouthed pipette tips. DNA was precipitated from the supernatant by incubation in 95% ethanol on ice for 1 h. DNA threads are spooled onto a glass hook, washed in 75% ethanol containing 0.2 M sodium acetate for 10 min, air-dried for 5 min and resuspended in TE buffer. Five μl RNAse A was added to the samples and incubated at 37° C. for 1 hour.

A combination of TAQMAN® and KASPar assays at 168 genome-wide SNPs were used to genotype the mapping population and create linkage groups. Map Manager QTX.b20 (Manly et al. (2001) Mammalian Genome 12:930-932; available online at mapmanager.org) was used to construct the linkage map and to perform a QTL analysis. The initial parameters were set at: Linkage Evaluation: Intercross; search criteria: p=1e⁻⁵; map function: Kosambi; and, cross type: line cross. These 168 markers formed 30 linkage groups across 19 chromosomes, with 17 markers unlinked. Marker regression (p=0.01) done on markers from LG A1 confirmed a significant QTL on this linkage group. A permutation test using 1000 iterations was used to establish the threshold for QTL significance (logarithm of odds (LOD) ratio statistic (LRS)). The permutation test determined that an LRS of at least 4.9 was suggestive, at least 11.0 was significant, and at least 18.3 was highly significant. Interval mapping was performed using the bootstrap test, free regression model, and the LRS cutoffs determined in the permutation test.

A total of 26 markers previously identified on LG A1 formed a single linkage group. Marker regression and interval mapping analysis (Table 2) completed using MapManager QTX.b20 indicated that the eight polymorphic SNPs on LG A1 are all tightly associated with the iron deficiency tolerance trait (Likelihood Ratio Statistic: 4.8-52.1, Percent Variation Explained: 2-18%). The interval of significance spanned a region from beyond marker S00405-1 to S01282-1. The region of significance included public markers BARC-044481-08709 (9097270 bp, 22.52 cM) and BARC-019031-03052 (7546740 bp, 14.63 cM) at ˜14-23 cM and sat_{—}137 (995905 bp, 3.63 cM).

TABLE 2

| | | PVE | | |
|---|---|---|---|---|
| Marker | LRS | (%) | Genetic (cM, v 4.0) | Physical (bp) |
| S15122-001-Q001 | 48.3 | 17 | 27.93 | 8,659,968 |
| S15124-001-Q001 | 42.2 | 16 | 27.93 | 8,671,038 |
| S15126-001-Q001 | 48.0 | 17 | 27.95 | 8,796,827 |
| S00405-1-A | 52.1 | 18 | 27.95 | 8,810,680 |
| S14531-001-Q001 | 16.3 | 6 | 2.45 | 620,718 |
| S01261-1-A | 15.0 | 6 | 2.45 | 620,718 |
| S01282-1A | 12.1 | 5 | 13.45 | 2,012,649 |
| S10245-1-K1 | 4.8 | 2 | 14.85 | 2,573,680 |

### Example 2

An F2 population comprising 368 progeny was developed by crossing 90M01 (TOL) with 92M01 (SUS). Genomic DNA from each progeny was isolated for analysis as described in Example 1 and used to genotype each sample.

Plants were phenotyped as described in Example 1 to generate a best linear unbiased prediction (BLUP) score phenotype dataset from 361 progeny used for analyses. The phenotypic dataset showed normal distribution across the score space.

Map Manager QTX.b20 (Manly et al. (2001) Mammalian Genome 12:930-932; available online at mapmanager.org) was used to construct the linkage map and to perform QTL analysis. The initial parameters were set at: Linkage Evaluation: Intercross; search criteria: p=1e⁻⁵; map function: Kosambi; and, cross type: line cross. A permutation test using 1000 iterations was run using the free model for each set of phenotypic data to establish the threshold for QTL significance (LRS). The permutation test determined that an LRS of at least 4.2 was suggestive, at least 10.8 was significant, and at least 19.0 was highly significant. Interval mapping was performed using the bootstrap test, free regression model, and the LRS cutoffs determined in the permutation test.

A total of 115 TAQMAN® and KASPar markers were used to genotype the population groups. These markers formed 16 linkage groups, with 22 markers unlinked. A total of 33 markers making up one linkage group were located on LG A1. Marker regression and interval mapping analysis (Table 3) completed using MapManager QTX.b20 indicated a highly significant ATL near the top of LG A1 tightly associated with the iron deficiency tolerance trait. Several polymorphic SNP markers were discovered in the region, these had LRS ranging from about 20-79.8, and indicated Percent Variation Explained (PVE) ranging from about 5-20%). The interval was tightly linked to S12985-1 (LRS=79.8; 20% PVE), and markers S15124-001 and S15121-001 explain about 15% of the phenotypic variation (LRS=57.3).

TABLE 3

| | | PVE | | |
|---|---|---|---|---|
| Marker | LRS | (%) | Genetic (cM, v 4.0) | Physical (bp) |
| S05017-1-K1 | 59.2 | 15 | 28.0 | 9,097,414 |
| S07022-1-K001 | 60.1 | 15 | 27.98 | 9,002,798 |
| S10456-1-K1 | 54.9 | 14 | 27.95 | 8,796,827 |
| S15071-001-Q001 | 53.6 | 14 | 27.95 | 8,796,827 |
| S15124-001-Q001 | 57.3 | 15 | 27.93 | 8,671,038 |
| S13062-1-K1 | 77.6 | 19 | 27.92 | 8,622,812 |
| S12985-1-K1 | 79.8 | 20 | 27.93 | 8,659,986 |
| S13064-1-K1 | 48.2 | 12 | 27.85 | 8,173,288 |
| S05933-1-K1 | 47.4 | 12 | 27.81 | 7,943,632 |
| S13078-1-K1 | 46.6 | 12 | 27.80 | 7,850,805 |
| S13073-1-K1 | 48.8 | 13 | 27.77 | 7,677,721 |

### Example 3

Marker S04776-1 is also associated with iron deficiency tolerance and can be used for selection of the LG A1 FeC QTL. For example, while the marker worked predictably for the majority of lines tested, lines where the S00405-1 allele did not predict the phenotypic effects of the QTL were observed, such as the proprietary soybean variety 91B42 (U.S. Pat. No. 6,855,874) and its descendents. In these cases, marker S04776-1 association with iron deficiency tolerance was confirmed in a survey of 183 soybean lines which included proprietary and public varieties, and is located at about 27.83 cM on the latest public genetic map (v 4.0). From these 183 varieties, 81 were homozygous for allele G, 101 were homozygous for allele C, and 1 was heterozygous. This analysis confirmed that allele G at position Gm05 8021614 is associated with improved iron deficiency tolerance.

### Example 4

Two F2 populations, 92M01×90M60 and 90M60×92M01, were evaluated by a genome-wide scan to identify QTLs conferring resistance to iron chlorosis. The populations consisted of 384 progeny each. DNA was isolated as described in Example 1. A set of 202 polymorphic markers were selected across all 20 chromosomes using a proprietary software, and the samples were genotyped. Phenotypic datasets were BLUP scores for 130 progeny of 92M01×90M60, and 147 progeny from 90M60×92M01. The phenotypic distribution for each population showed a normal distribution. MapManager QTX.b20 was used to construct the linkage map with initial parameters set at: Linkage Evaluation: Intercross; search criteria: p=1e⁻⁵; map function: Kosambi; and, cross type: line cross. Single marker analysis, composite interval mapping, and multiple interval mapping were executed using QTL Cartographer 2.5 (Wang et al. (2011) Windows QTL Cartographer 2.5; Dept. of Statistics, North Carolina State University, Raleigh, N.C. Available online at statgen.ncsu.edu/qticart/WQTLCart.htm). The standard CIM model and forward and backward regression method was used, and the LRS threshold for statistical significance to declare QTLs was determined by a 500 permutation test.

The allele calls from genotyping data were converted to the A (maternal), B (paternal), H (heterozygous) convention for mapping analysis. For population 92M01×90M60, 7 markers were removed returning more than 30% missing data, and 95 markers showed severe segregation distortion (p<0.0001). Nearly all distorted markers (94%) were skewed heavily toward the 92M01 allele, with the average ratio of approximately 11A:1H:3B. In the 90M60×92M01 population, 8 markers were identified as missing more than 30% data, and 29 were severely distorted. No progeny were identified in either population as selfs. In addition to examining each population individually, the data sets were combined assigning 92M01 as parent A and 90M60 as parent B. Eight markers were missing more than 30% data and were removed from the analysis. 102 markers were severely distorted, with 88 skewed heavily toward the 92M01 allele.

The linkage maps were constructed using non-distorted markers to create a frame-work, and then distorted markers were distributed into the linkage groups where possible. Marker order was checked against a standard benchmark map to verify that distorted markers were distributed to the correct locations. For population 92M01×90M60, 82 non-distorted markers formed 29 linkage groups. Four markers showing segregation distortion were then distributed into the linkage groups. In total, 109 markers remained unlinked. For population 90M60×92M01, 160 non-distorted markers formed 34 linkage groups and five distorted markers were successfully distributed. 29 markers remained unlinked. 108 non-distorted markers and 18 distorted markers formed 44 linkage groups using the combined genotypic data, while 67 markers remained unlinked. The linkage map and cross data for each data set was exported in QTL Cartographer format for subsequent analysis.

Single marker analysis showed a highly significant association in each population at marker S05933-1-Q1 near the top of LG A1 (7943632 bp, 27.81 cM), explaining up to 29.3% of the phenotypic variation, with the effect coming from parent 90M60. The marker was unlinked in both populations and significance could not be confirmed by composite interval mapping, however this marker is within about 2 cM of S15124-001-Q001.

### Example 5

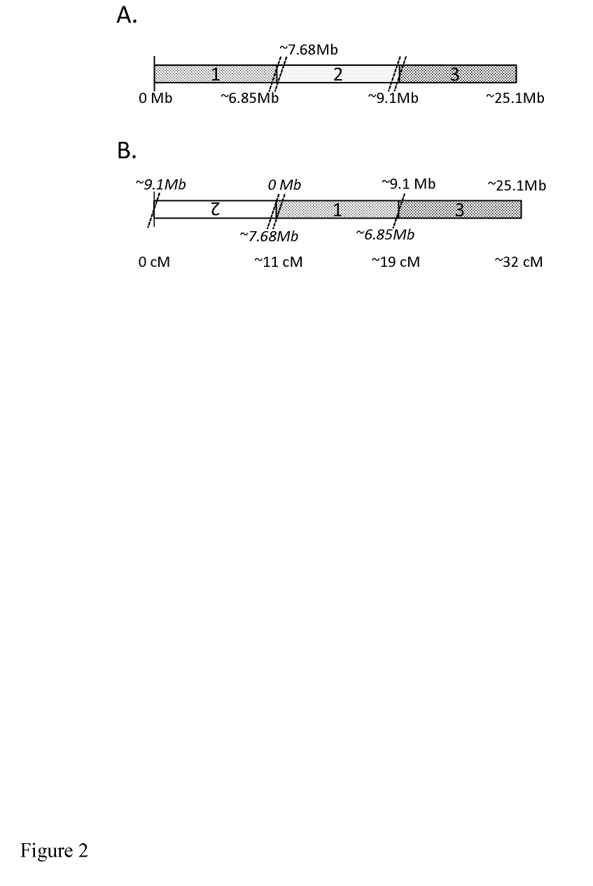

Analysis of the physical location of the assayed SNPs on LG A1 in relation to their estimated genetic linkage relationships in the two populations suggests a misassembly in the physical Glyma1.0 reference. Based on linkage analyses completed herein, a small region containing marker S00405-1 (8810680 bp) should physically exist near the telomere of chromosome 5 approximately 18 cM from S14531-001 (620718 bp), 29 cM from marker S01282-1 (2012649 bp) and over 50 cM from S14561-001 (3603395 bp). This would place the iron chlorosis tolerance QTL at the tip of chromosome 5 on the physical map and below 0 cM on the public 2010 consensus genetic map. Our data also suggests that this region is inverted as compared to available public physical and genetic maps. We estimate this region to include the region containing BARC-044481-08709 (9097471 bp) and BARC-019031-03052 (7546385 bp) at ˜24-25 cM on the 2003 public consensus map. A corrected map order and estimated positions is provided in Table 5. The borders of the misassembled sequence appear to be located between S05107 (6,852,084 bp) and S13073 (7,677,721 bp) and between S05017 (9,097,414 bp) and S01462 (25,074,885 bp). These putative borders are shown in italics in Table 5. The misassembly translocates and inverts the order of the top of LG A1 as compared to the mapping population data described above. This data is also summarized in FIG. 2.

TABLE 5

| Marker | Genetic (cM, | Physical | Mapping Study - |
|---|---|---|---|
| order | v 4.0) | (bp)* | est. position (cM) |
| S15081 | 27.94 | 8,712,346 | 0 |
| S00405 | 27.95 | 8,810,680 | 3.3 |
| S05017 | 28.0 | 9,097,414 | 6 |
| S07022 | 27.98 | 9,002,798 | 6.2 |
| S10456 | 27.95 | 8,796,827 | 7.2 |
| S15126 | 27.95 | 8,809,479 | 7.2 |
| S15071 | 27.95 | 8,796,827 | 7.2 |
| S15124 | 27.93 | 8,671,038 | 8.6 |
| S15121 | 27.93 | 8,650,576 | 8.6 |
| S15122 | 27.93 | 8,659,968 | 11.1 |
| S13062 | 27.92 | 8,622,812 | 11.1 |
| S15125 | 27.93 | 8,673,968 | 11.2 |
| S15123 | 27.93 | 8,660,316 | 11.2 |
| S12985 | 27.93 | 8,659,986 | 11.2 |
| S13064 | 27.85 | 8,173,288 | 15.4 |
| S05933 | 27.81 | 7,943,632 | 17.1 |
| S13078 | 27.80 | 7,850,805 | 17.8 |
| S13073 | 27.77 | 7,677,721 | 19.4 |
| S01261 | 2.45 | 620,718 | 31.2 |
| S14531 | 2.45 | 620,718 | 31.3 |
| S01282 | 13.45 | 2,012,649 | 43.2 |
| S14582 | 14.88 | 2,578,312 | 51.1 |
| S10245 | 14.85 | 2,573,680 | 51.3 |
| S14581 | 15.48 | 2,703,606 | 53.3 |
| S10446 | 18.40 | 3,271,804 | 59.9 |
| S14561 | 19.98 | 3,603,395 | 64.8 |
| S14552 | 19.99 | 3,604,317 | 64.8 |
| S14562 | 19.94 | 3,597,393 | 64.9 |
| S13012 | 26.41 | 5,711,938 | 75.3 |
| S05107 | 27.64 | 6,852,084 | 76.9 |
| MarkerA | 29.56 | 25,074,885 | 80.3 |

*Glyma 1 assembly

The markers identified by Charlson et al. (2003 J Plant Nutr 26:2267-2276) and Lin et al. (1997 Mol Breed 3:219-229; and 2000 J Plant Nutr 23:1929-1939) as positioned by Mamidi et al. (2011 Plant Genome 4:154, and Supplemental Table 1) are found on the consensus soybean genetic map as follows:

| | | | |
|---|---|---|---|
| Lin | K258-A256 | BARC-050075-09365 | 31.37 cM |
| Charlson | Satt211 | BARC-058665-17450 | 80.46 cM |

Correcting the misassembly will shift them further away from the top of LG A1 (ch5), with their corrected positions to be determined.

Mamidi et al. (2011 Plant Genome 4:154, and Supplemental Table 1) did not find a significant FeC marker on LG A1 in their association studies, however BLAST analysis with Arabidopsis protein genes involved in iron metabolism did identify BARC-053261-11776 as a neighbor of a putative AHA2 gene homolog at Glyma05g01460, which starts at 960,820 bp. Correcting the misassembly will shift this region away from the top of LG A1 (ch5), with the corrected positions to be determined.

### Example 6

From the analyses of marker loci associated with iron deficiency tolerance in soybean populations and varieties several markers were developed, tested, and confirmed, as summarized in preceding tables. Any methodology can be deployed to use this information, including but not limited to any one or more of sequencing or marker methods.

In one example, sample tissue, including tissue from soybean leaves or seeds can be screened with the markers using a TAQMAN® PCR assay system (Life Technologies, Grand Island, N.Y., USA).

TAQMAN ® Assay Conditions
Reaction Mixture (Total Volume = 5 μl):

| | | |
|---|---|---|
| Genomic DNA (dried) | 16 | ng |
| DDH20 | 2.42 | μl |
| Klearkall Mastermix | 2.5 | μl |
| Forward primer (100 μM) | 0.0375 | μl |
| Reverse primer (100 μM) | 0.0375 | μ |
| Probe 1 (100 μM) | 0.005 | μl |
| Probe 2 (100 μM) | 0.005 | μl |

Reaction Conditions:

| | |
|---|---|
| 94° C. | 10 min 1 cycle |

40 cycles of the following:

| | | |
|---|---|---|
| 94° C. | 30 | sec |
| 60° C. | 60 | sec |

Klearkall Mastermix is available from KBioscience Ltd. (Hoddesdon, UK).

A summary of the tolerant and susceptible alleles for iron deficiency markers is provided in Table 6.

TABLE 6

| | | | Allele |
|---|---|---|---|
| Marker | Genetic (cM) | Physical (bp) | (tol/sus) |
| S00405 | 27.95 | 8,810,680 | G/C |
| S15121 | 27.93 | 8,650,576 | T/A |
| S15124 | 27.93 | 8,671,038 | A/G |
| S04776 | 27.83 | 8,021,614 | G/C |
| S15081 | 27.94 | 8,712,346 | —/T |
| S05017 | 28.0 | 9,097,414 | A/G |
| S07022 | 27.98 | 9,002,798 | T/C |
| S10456 | 27.95 | 8,796,827 | A/G |
| S15126 | 27.95 | 8,809,479 | A/G |
| S15071 | 27.95 | 8,796,827 | A/G |
| S15122 | 27.93 | 8,659,968 | G/A |
| S13062 | 27.92 | 8,622,812 | C/G |
| S15125 | 27.93 | 8,673,968 | T/C |
| S15123 | 27.93 | 8,660,316 | A/G |
| S12985 | 27.93 | 8,659,986 | A/G |
| S13064 | 27.85 | 8,173,288 | T/C |
| S05933 | 27.81 | 7,943,632 | A/C |
| S13078 | 27.80 | 7,850,805 | G/C |
| S13073 | 27.77 | 7,677,721 | T/A |
| S01261 | 2.45 | 620,718 | A/T |
| S14531 | 2.45 | 620,718 | T/A |
| S01282 | 13.45 | 2,012,649 | G/A |
| S14582 | 14.88 | 2,578,312 | C/T |
| S10245 | 14.85 | 2,573,680 | G/A |
| S14581 | 15.48 | 2,703,606 | T/A |
| S10446 | 18.40 | 3,271,804 | A/G |
| S14561 | 19.98 | 3,603,395 | T/A |
| S14552 | 19.99 | 3,604,317 | G/C |
| S14562 | 19.94 | 3,597,393 | G/A |
| S13012 | 26.41 | 5,711,938 | T/C |
| S05107 | 27.64 | 6,852,084 | T/C |

A summary of marker sequences is provided in Tables 7 and 8.

TABLE 7

| | | SEQ | | SEQ |
|---|---|---|---|---|
| Marker | Primers (FW/REV) | ID | Probes | ID |
| S00405 | GGGTTGAGTTGCAGAGGCTGAA | 1 | CTTCAcCTAAATCAG | 3 |
| | ATGACCATGAATGGGTCCAAGC | 2 | CTTCAgCTAAATCAG | 4 |
| S15121 | CCAATTAAAATTTTCCTCAGATCC | 6 | cagcatcTcaggcca | 8 |
| | TA | | | |
| | TGGTTTGATGGGTGCTTAATTT | 7 | cagcatcAcaggcc | 9 |
| S15124 | TCGATCTCAAAATGCTAAATCTTG | 11 | tctcattcaAgtcccctg | 13 |
| | T | | | |
| | TTGTGCATTGAAAGTTTTGACTTC | 12 | tcattcaGgtcccc | 14 |
| | TT | | | |
| S04776 | acccgggaatgtgaatgata | 16 | aagacatCattgctca | 18 |
| | tatccaatggtggggatgg | 17 | aagacatGattgctca | 19 |
| S15081 | GTTTATCCAGACTGCTTTCTTTTG | 21 | tctgacagccttctt | 23 |
| | T | | | |
| | TGCTCCTAGATAGGAATATTTAAC | 22 | cagccttCTTActtgtta | 24 |
| | ACG | | | |
| S05017 | GCATTAATAGCCACCTCGCACCTA | 26 | GAAGGTGACCAAGT | 27 |
| | A | | TCATGCTTTGTGGAT | |
| | | | GAAAAGCAAGAGC | |
| | | | GC | |
| | | | GAAGGTCGGAGTCA | 28 |
| | | | ACGGATTCTTTGTG | |
| | | | GATGAAAAGCAAGA | |
| | | | GCGT | |
| S07022 | CATGCCTCCTGAATCCCAATAGCT | 30 | GAAGGTGACCAAGT | 31 |
| | T | | TCATGCTATGTCAA | |
| | | | TACTTGGAGTACAC | |
| | | | ATCATC | |
| | | | GAAGGTCGGAGTCA | 32 |
| | | | ACGGATTCATGTCA | |
| | | | ATACTTGGAGTACA | |
| | | | CATCATT | |
| S10456 | AAAATTCTATGGAAATGGATCCC | 34 | GAAGGTGACCAAGT | 35 |
| | CTACATT | | TCATGCTGAGCGAT | |
| | | | TTTCATCCAGCAGTT | |
| | | | TTA | |
| | | | GAAGGTCGGAGTCA | 36 |
| | | | ACGGATTGAGCGAT | |
| | | | TTTCATCCAGCAGTT | |
| | | | TTG | |
| S15126 | GCATGCAAGATCTAAACTGAGC | 38 | ccagcagttttAcacaa | 40 |
| | TGGAAATGGATCCCCTACAT | 39 | ccagcagttttGcaca | 41 |
| S15071 | AAAATAAGCATGCAAGATCTAAA | 43 | ccagcagttttAcac | 45 |
| | CTG | | | |
| | ATGGAAATGGATCCCCTACA | 44 | ccagcagttttGcac | 46 |
| S15122 | CATCGATTATTCCCACAAACC | 48 | ctcacacGctttct | 50 |
| | ACTAGTTATGTGATGGTTGATCTT | 49 | tcacacActttctc | 51 |
| | CTG | | | |
| S13062 | GGGACACGTTAATCAGGGACAAA | 53 | GAAGGTGACCAAGT | 54 |
| | GTT | | TCATGCTAAACGGT | |
| | | | TCAGATCAAAACAC | |
| | | | GTGC | |
| | | | GAAGGTCGGAGTCA | 55 |
| | | | ACGGATTAAACGGT | |
| | | | TCAGATCAAAACAC | |
| | | | GTGG | |
| S15125 | GCTTGATGATTTAGATTCGAACTG | 57 | ttgacaatttaacTgatcc | 59 |
| | TTAT | | | |
| | TCAAGACCTATTTTGCGCTTT | 58 | acaatttaacCgatccta | 60 |
| S15123 | aaggcatgcatagcactttaact | 62 | aatacccTggttgaatc | 64 |
| | ccatcttcaattgtacagtttcatactt | 63 | atacccCggttgaat | 65 |
| S12985 | TAGTTATGTGATGGTTGATCTTCT | 67 | GAAGGTGACCAAGT | 68 |
| | GGGAA | | TCATGCTTTTCAACA | |
| | | | TGTTTTATCCTTACT | |
| | | | CACACA | |
| | | | GAAGGTCGGAGTCA | 69 |
| | | | ACGGATTCAACATG | |
| | | | TTTTATCCTTACTCA | |
| | | | CACG | |
| S13064 | GGCTGTGTTGAGGGTGGAGGAT | 71 | GAAGGTGACCAAGT | 72 |
| | | | TCATGCTGATCCAA | |
| | | | ACCCAACGTAACCT | |
| | | | GG | |
| | | | GAAGGTCGGAGTCA | 73 |
| | | | ACGGATTAGATCCA | |
| | | | AACCCAACGTAACC | |
| | | | TGA | |
| S05933 | ATGAGAAGAAATGGACTGATGGA | 75 | GAAGGTGACCAAGT | 76 |
| | AGTGTT | | TCATGCTCAAGCCT | |
| | | | TGCAAGGTTCCCAG | |
| | | | AA | |
| | | | GAAGGTCGGAGTCA | 77 |
| | | | ACGGATTAAGCCTT | |
| | | | GCAAGGTTCCCAGA | |
| | | | C | |
| S13078 | CCATTTCTGAATCAACAGACGCCC | 79 | GAAGGTGACCAAGT | 80 |
| | AA | | TCATGCTGGAAAAT | |
| | | | CATTGTTAGTTACCT | |
| | | | GATCTAG | |
| | | | GAAGGTCGGAGTCA | 81 |
| | | | ACGGATTGGAAAAT | |
| | | | CATTGTTAGTTACCT | |
| | | | GATCTAC | |
| S13073 | AATCTTGATTCCTATTTGGGTTTC | 83 | GAAGGTGACCAAGT | 84 |
| | CTWGTA | | TCATGCTAATATGG | |
| | | | GGTTTAAACAGCTA | |
| | | | CTCATA | |
| | | | GAAGGTCGGAGTCA | 85 |
| | | | ACGGATTCTAATAT | |
| | | | GGGGTTTAAACAGC | |
| | | | TACTCATT | |
| S01261 | aaagaccagcactccagcat | 87 | tagatcctccAttttt | 89 |
| | tagaggaaagggtggtggtg | 88 | atcctccTtttttcc | 90 |
| S14531 | aaagaccagcactccagcat | 92 | atgtttagatcctccAttt | 94 |
| | tagaggaaagggtggtggtg | 93 | atgtttagatcctccTtt | 95 |
| S01282 | tgcacacacacccaatcac | 97 | aagagagaatccaGttga | 99 |
| | accttctaatcccgcctctt | 98 | aaaagagagaatccaAttg | 100 |
| S14582 | agatcaatggcacccttacg | 102 | tccagtgacttttgCac | 104 |
| | ttccagatccagatagcaacttc | 103 | ttccagtgacttttgTac | 105 |
| S10245 | CAGAAAGAAGAGCACCACCAACC | 107 | GAAGGTGACCAAGT | 108 |
| | AA | | TCATGCTATCCACTC | |
| | | | CCTTTCCTGTTCCTA | |
| | | | GAAGGTCGGAGTCA | 109 |
| | | | ACGGATTCCACTCC | |
| | | | CTTTCCTGTTCCTG | |
| S14581 | TGGCTGGTTCGTACAATCG | 111 | tctcatcTcaattcaa | 113 |
| | GGAGCGAGGTCAAAGAGAAGTA | 112 | tctcatcAcaattca | 114 |
| S10446 | caagccgacatcggaaaa | 116 | caacgtcActtgaaa | 118 |
| | cgacattgtccagggctatt | 117 | acgtcGcttgaaaa | 119 |
| S14561 | tccggtatcggtttataagtttg | 121 | ctgatccaaTccaaac | 123 |
| | acgattgtgctgaagtgctg | 122 | ctgatccaaAccaaac | 124 |
| S14552 | CGTCCAGCCACACACAAC | 126 | aattgcttcaCgtttca | 128 |
| | GAAATCATCAACAAGTGATCATC | 127 | ttgcttcaGgtttcat | 129 |
| | C | | | |
| S14562 | ttttaggtttgactgatcttggaa | 131 | tagaactCagagaccc | 133 |
| | aatttctttgccatgcaagtg | 132 | attagaactTagagaccc | 134 |
| S13012 | AGCTGTGGCTTACTAACATTAGG | 136 | cttgcaaacTtggatc | 138 |
| | G | | | |
| | ATTTAAACCTATCCAAATCAACTA | 137 | cttgcaaacCtggat | 139 |
| | CG | | | |
| S05107 | CAATGGCCGACATCCAC | 141 | caccacatTcaac | 143 |
| | AACAATGCATGTGATAGAATAAA | 142 | caccacatCcaac | 144 |
| | AGC | | | |

TABLE 8

| | | SEQ |
|---|---|---|
| Marker | Genomic region | ID |
| S00405 | tYcYBBKDDWWMHRGGCYWTGACYWTKWATKKGTYCA | 5 |
| | AGCTCGAAGAAAGCTTCASCTAAATCAGGTGTGACAAA | |
| | KCACATGAAGGGCTTCAGCCTCTGCAACTCAACCCTAAA | |
| | AGTAAATGGGCAAGTCATCCTCTCCCAAGTCCCCAAGA | |
| | ACGTAACCCTCACCCCATGCACCTACGACACTCACACCA | |
| | CCGGATGCTTCCTCGGTTTCCACGCCACCTCCCCAAAAT | |
| | CCCGCCACGTGGCACCCTTAGGACAGCTTAAAAACATA | |
| | AGCTTCACTTCCATCTTCCGGTTCAAGGTTTGGTGGACC | |
| | ACTCTCTGGACCGGCTCCAACGGCCGCGACCTGGAAAC | |
| | CGAAACCCAATTCCTCATGCTCCAATCCCACCCTTATGT | |
| | TCTCTTCCTACCCATCCTCCAACCCCCATTTCGCGCCTCG | |
| | CTGCAGCCTCACTCAGACGACAACGTTGCGGTGTGTGTG | |
| | GAGAGCGGCTCCADCCRSGTAACAGCCTCATCATTCGAC | |
| | ACTGTCGTCTACTTGCACGCAGGGGACAACCCWKSc | |
| S15121 | TTCACATCAAAATTTTGTACTTTCCTAATTCTGCTGGTCC | 10 |
| | GTACTGACGGATTTTTACCGTTTTAAATAACTGTAATGG | |
| | TGGTGTGTGTAAGAATGGGACAGTCAGTGGCCAAAAAT | |
| | CCAAGAATCAATTATCCACCCAACCACCAATTAAAATTT | |
| | TCCTCAGATCCTAGGGTCCCATGATTCCAACCAAAAGCA | |
| | GCATCWCAGGCCAAAAAATTAAGCACCCATCAAACCAC | |
| | TTTTTAGCTTTTCCAACTTATGTTCCTCTTCCCCCCTGCA | |
| | AAAATAATGTCTAGACCACTGCAGGCTCTCGGAGCTTTT | |
| | TCTGAAGATTATGTGCCACAGCATCAATAAATTCCTCCG | |
| | TGTTCAAGTAAAATTCCCTTGATACCCTGCACATCATGC | |
| | ATGCTCGGGTG | |
| S15124 | CCGAAACCTCCCAAGTACCAGTAATTTTAATACGTAGTG | 15 |
| | CTGCAGAATGTGATGAAGAGGAGAAATCATCCAGCTTG | |
| | AATCTGTCTTCAAAAAATCCTCAAGTAGATAATGGGGTG | |
| | CAATTGGATCTCAAGTCAAAATCTCGATCTCAAAATGCT | |
| | AAATCTTGTAGATCTCGAGATGTTGATGCTCCATCTTCT | |
| | CATTCARGTCCCCTGCCTTATACAAATGTTAAGAAGTCA | |
| | AAACTTTCAATGCACAAAGAGTCAAAATCTGATCTTCAA | |
| | AGGCCAAAAGGGGATGAGCAGGGACCTAAAGATAAGG | |
| | TAACTGCAKAAGATCTGAAACTCGGAAGCGAAGTAACT | |
| | GCCAAAGTCTCGCAAATTGGTGCACATGGGTTAGTGTTG | |
| | GATTTGGGTGGAGGA | |
| S04776 | AATTTCTGAAACTCAGTAGCAACGTTCTACATGAATTTT | 20 |
| | CTATTTAAAGATTCACAAATGTAATAAGCCACTTGTCAT | |
| | AGCATGGTATACCAAGGCAGAGTTTGGTAACACTAGCT | |
| | AAGTAGCCATGCGWTTTACACAGATACTAACAGAATAA | |
| | GACATAAGAACAGAATGTGAGGAATATTTTAGGGAGGC | |
| | TCTGAATCAATGTTAGTTTGAGGAATGCAATTTCCTTAT | |
| | TCATGACTTATTTTKGGTAGGGTTGTCACATTACCCGGG | |
| | AATGTGAATGATACTGACATTGTAAAAAGACATSATTGC | |
| | TCACACCCTTAATGATCTAGCCCCCCCAATGCCCCATCC | |
| | CCACCATTGGATAGCCTTCACTACCACTGTCAATGTCAC | |
| | TGTCACTTCCATCACTCAATAAATTGTCATTAGGTTGAA | |
| | ATTTTGAAGAAAAAAAAAATCCnATTTTCCCTCnTTAnCC | |
| | TATTTTTGGTCtCAATTT | |
| S15081 | aTCTAATTGGCTAAAACTAAAACACTAATCTAAGGTGGC | 25 |
| | TAATGGCTACACCTTGTTGCTTTCATCCTAGGATAGGCC | |
| | AACTCCCTAGAGGCAGCTCCATCTAATGGGCGAAAACTT | |
| | ATGTCTCAAGGGACGGCTTCATCATGTTGGTAGTCTTTG | |
| | TTTGCCTCTCACAGGCGGCACTTTGCTCCTAGATAGGAA | |
| | TATTTAACACGACACATTCTAACAAGTAAGAAGGCTGTC | |
| | AGACAACAAAAGAAAGCAGTCTGGATAAACATTAAACA | |
| | TATTATCTTCCATTTCTACAAGATTACGTTAYGAAAGCA | |
| | ATAACTTGCCTGCAATAAACAAGATGGAATTTGGATATA | |
| | ACCCTGTAGCAAGACGTTGACTTTTTCGAGAGAATTATG | |
| | TGGTACAGGAGTAATGACATAAAGCACACCTTTGACTGT | |
| | GTCAATTCCCCTCACAATCCCTGAAACATAAGAAATCAA | |
| | GATGTGTTTAACAATTCATATGCTTAGAATTATTGAAAT | |
| | GCATAACTACTCAACTAGAACAGTATCTGTATGATTTGT | |
| | TTCCATCATGGATACACTTTTCTTTTTTTATCTTTTACAT | |
| | TTATGGAAGTATGATATTACAAAGCTAAGAGACACTGGT | |
| | AAACTTGTTACAACCAATATAGACaGACATACnAGATCT | |
| | AATGAAGCWGTGAAATTTAAGGACTACaTTGTTGAAGA | |
| | TTCTGAATTAAATTACATTACTGAAGTGCTGAGTTAAGT | |
| | TCTCAGCATATCTcAnTATACATATa | |
| S05017 | TGGTGATGAAGAAGACGACGACGGCGGTGGCTCCTTTG | 29 |
| | TGGATGAAAAGCAAGAGCGYTAGGGGTGGTATTAGGTG | |
| | CGAGGTGGCTATTAATGCGGTGGATGAGTCGACGACGT | |
| | CACCGGAGKCGAAGATTGGAGCGCGTGTGAAGGTGAAG | |
| | GCGGGTGTAAAGGTGTACCACGTCCCCAAAGTAGCCGA | |
| | GCTTGACCTCACGGGTCTGGAAGGCGAGATCAAGCAGT | |
| | ATGTTGGCCTCTGGAACGGTAAGCGAATCTCCGCCAATC | |
| | TTCCTTACAAGGTTCAGTTTCTCACCGACATCCCAGCTC | |
| | GTGGTCCT | |
| S07022 | gGSCTAGCTTTTATCTTTGAAGAnACTACTACTAAGTTGA | 33 |
| | ARTTAAAAAAAAATTCTAATGTTGTTTGGTATTTTATGG | |
| | TTTTTTAACTTAATTGCAAACTTCTACATTACTAGTGATT | |
| | GTCAAACAATTGGCATGCACTACAAACATGTCAATACTT | |
| | GGAGTACACATCATYAAGATTAGTGAGCACAAAGCTAT | |
| | TGGGATTCAGGAGGCATGGTTTTGAGAATGCATATGGA | |
| | GCTTGATACCCAGGAAAACATCACTTAACTAAGAGCAA | |
| | GCTTTGAGGATGATAGGATCGTGGCCGAGACTAARAAT | |
| | GAGCCTCATGAGGTAATAGAATGAAACAAGGGCATGCA | |
| | TATTGATGAGGAAATAGTAGCAGATCACAACTTTGAAG | |
| | GAATAGAAATTGGGCGAGAGCAGGTGGTTGAACAAGAG | |
| | ATCACCAAAGAGGAGCTTTGTGACAAACATGGAGGTTG | |
| | TCAATCAGGCCATGCATGGTCATAGCTKK | |
| S10456 | GAATCTCCTACTTGCCATGCCACAACAAGCTATAGAATT | 37 |
| | TTGGATAGGGATTCCTTGCAGTCACAGATTTTCAGCATT | |
| | CACACATTCAGCTACTCAAAATCGTTGGATGTAGATCTG | |
| | ACAACTCATATTTTATGCCGATAAACCTAACTTAAAAAT | |
| | AAGCATGCAAGATCTAAACTGAGCGATTTTCATCCAGCA | |
| | GTTTTRCACAAATGTATGAATGTAGSGGATCCATTTCCA | |
| | TAGAATTTTATGGTTAACCACCCAAGCTGAAAAGTGGG | |
| | GATTTGATAGAAACGACAAAGGGGCCTGACCCAACTCC | |
| | CAATGCTGAACCCCCAGATTTCACAGAACATACCTTCCA | |
| | TTTCCTCTAAGCACTTGCCAATATGCATAATTTTAAAAA | |
| | ATCAAGCAGCAAT | |
| S15126 | GAATCTCCTACTTGCCATGCCACAACAAGCTATAGAATT | 42 |
| | TTGGATAGGGATTCCTTGCAGTCACAGATTTTCAGCATT | |
| | CACACATTCAGCTACTCAAAATCGTTGGATGTAGATCTG | |
| | ACAACTCATATTTTATGCCGATAAACCTAACTTAAAAAT | |
| | AAGCATGCAAGATCTAAACTGAGCGATTTTCATCCAGCA | |
| | GTTTTRCACAAATGTATGAATGTAGGGGATCCATTTCCA | |
| | TAGAATTTTATGGTTAACCACCCAAGCTGAAAAGTGGG | |
| | GATTTGATAGAAACGACAAAGGGGCCTGACCCAACTCC | |
| | CAATGCTGAACCCCCAGATTTCACAGAACATACCTTCCA | |
| | TTTCCTCTAAGCACTTGCCAATATGCATAATTTTAAAAA | |
| | ATCAAGCAGCAAT | |
| S15071 | TTTACACACAAAAATAAAGAAATTTTTGGAACTTGAATC | 47 |
| | TCCTACTTGCCATGCCACAACAAGCTATAGAATTTTGGA | |
| | TAGGGATTCCTTGCAGTCACAGATTTTCAGCATTCACAC | |
| | ATTCAGCTACTCAAAATCGTTGGATGTAGATCTGACAAC | |
| | TCATATTTTATGCCGATAAACCTAACTTAAAAATAAGCA | |
| | TGCAAGATCTAAACTGAGCGATTTTCATCCAGCAGTTTT | |
| | RCACAAATGTATGAATGTAGGGGATCCATTTCCATAGAA | |
| | TTTTATGGTTAACCACCCAAGCTGAAAAGTGGGGATTTG | |
| | ATAGAAACGACAAAGGGGCCTGACCCAACTCCCAATGC | |
| | TGAACCCCCAGATTTCACAGAACATACCTTCCATTTCCT | |
| | CTAAGCACTTGCCAATATGCATAATTTTAAAAAATCAAG | |
| | CAGCAATCAAAGRGTTTTTCTATTATGCGCCAGCATCGG | |
| | CAGGTGCACGATAAAAAGTCAATAATAGAAACAAAAAT | |
| | CATTAGCAAAGAGAGCATTTATCAGATTGAAAACACAA | |
| | AAGTCACTAAATGCTTGCATTTGTTGAGTGATTTAAAAC | |
| | ATTCAACATATATATTTCATGAACACACGCACACACAGT | |
| | AGAGAACCCCATACTGAAAAAAACTAAAGTGAAATAGT | |
| | GTGTGAGTGTGGTGTGWGTGTGTGTGTGTATATTCTCCC | |
| | CCAGCATTGAAAGATAGCAAACACCCCCnTCGAGAGGA | |
| | CTCTCAAAATTATGGCATGGca | |
| S15122 | CCTCTCGRTGGTTCTCACATTATGATACTTCACTGAACAT | 52 |
| | CCTTGTTGGTCAAAACCCTYCACCAGTCAAAATTCTCTG | |
| | TAGATAGCCCTTTYTGAGACCTCGACAACCAACTCTGAC | |
| | AGTGCTCAACTGCTTTCAAGATCATCGATTATTCCCACA | |
| | AACCAACAAGAGATTTTTCAACATGTTTTATCCTTACTC | |
| | ACACRCTTTCTCGGAAACTTCCCAGAAGATCAACCATCA | |
| | CATAACTAGTCCAAGTCACCCACAAACCAATATGCTTCT | |
| | AAGATTAATGATTGTCCCACAAACCAACACAAGACTTTT | |
| | CAAYGTGATTTGTCCTCACTCACGTTTTTTGGAAAACTTT | |
| | CTAAAAGRTCACCCATCTCATAACTACTTCAAACCAAAC | |
| | ACGCTTAAT | |
| S13062 | AAAGAATTATTTATTTATTTTATATTTAAAAACCATTTAA | 56 |
| | ACATAATTTTGTAAAATAAAATTAATGTTATATTAACCC | |
| | TAATTCACTTTGTAGAGTATGATTTCTACATTTCTTTCCA | |
| | TTTCTTTTCCTTACTCCTCCCGAAAACAAAACCAGCTCTA | |
| | GAGCTGGTGTTGCAATCAAACGGTTCAGATCAAAACAC | |
| | GTGSACTGATTTGTGAGAAACTTTGTCCCTGATTAACGT | |
| | GTCCCCAGCGGGAGTGGAACACAGAAATTTTCACAGAA | |
| | AAAACTGCGCGAGTATGGAAAGTTTTTCCATGCTTGTGT | |
| | TGTTGCATTGTCCCTGAATTTGTACTTGATTCTGGGACGT | |
| | CACTCAGTCTGAGTCTGTTTCTGCAAATTTCAAAACCCT | |
| | CTCTCTCGT | |
| S15125 | TGGAATAAAATCTCTTCGTATACCCMTCTATAGTTTAGG | 61 |
| | TAAGACCAAAAGGGTCACTATAAATATTCACTATGATCG | |
| | GCTTAACGGTGGTGAATTGGAATAGATGCTTGATGATTT | |
| | AGATTCGAACTGTTATTATACACCAAAAAGATAAGGAT | |
| | CACTATGAATTCTCTGTAGTCTAATTGTTCTTTTGACAAT | |
| | TTAACYGATCCTAAAATATGAAAATATTAGATAAAAGC | |
| | GCAAAATAGGTCTTGATTCAGTTCTCTGTTAGTAACAAC | |
| | CTAAATTGCAAACACATCTCTAGTGTGAAWAGACATTG | |
| | ATACCTTAAAAAACACTAGTCAACCCAAAAGCWTGAGC | |
| | TTTGTAATCATTCATGTTTGTTATTGCCAATKTGGAATAT | |
| | TGTCCTTTTGGCA | |
| S15123 | ACTTTCTAAAAGRTCACCCATCTCATAACTACTTCAAAC | 66 |
| | CAAACACGCTTAATTGTGAAATTCTTAAGTGATAACTAC | |
| | CGAAAAGTAGATGCATCTTATTGGTATAAGAAATACCA | |
| | ATTAATTCTTTTAAACCATCTTCAATTGTACAGTTTCATA | |
| | CTTGTACAATCTCTGGATCCCCCTCATTCTGATGTGATTC | |
| | AACCRGGGTATTATACTCAAAAACAACAAGAATCAAGT | |
| | ATAAATAGTTAAAGTGCTATGCATGCCTTTTGTTTTTGCC | |
| | ACTTCCCATTGATTAAAAAAACTCACTTGAGAGTGGCTT | |
| | CAGCACTTTCCACAGTAACTCTGTCATCAGTAGCATCAC | |
| | GATTTTGAAGCCCCAAATCAAAGTACTTTATGTTCAAAT | |
| | CCAGGTAAGG | |
| S12985 | CCTCTCGGTGGTTCTCACATTATGATACTTCACTGAACA | 70 |
| | TCCTTGTTGGTCAAAACCCTTCACCAGTCAAAATTCTCT | |
| | GTAGATAGCCCTTTCTGAGACCTCGACAACCAACTCTGA | |
| | CAGTGCTCAACTGCTTTCAAGATCATCGATTATTCCCAC | |
| | AAACCAACAAGAGATTTTTCAACATGTTTTATCCTTACT | |
| | CACACRCTTTCTCGGAAACTTCCCAGAAGATCAACCATC | |
| | ACATAACTAGTCCAAGTCACCCACAAACCAATATGCTTC | |
| | TAAGATTAATGATTGTCCCACAAACCAACACAAGACTTT | |
| | TCAACGTGATTTGTCCTCACTCACGTTTTTTGGAAAACTT | |
| | TCTAAAAGGTCACCCATCTCATAACTACTTCAAACCAAA | |
| | CACGCTTAAT | |
| S13064 | TTATTGTATGGGGAGAATTGCACACCAGACCAGACAAG | 74 |
| | GGACCTAAACCTAACCCTGTGAACTGAAAATGGTGTGA | |
| | GGAAGAATTGGGATCGAAATGGTAAAAAGGAAAAGAA | |
| | AAGATATAAATATATTATTATAAAATTGGTAAAGAAAA | |
| | GGAGAAGAGGAAAGGAAGGCTGTGTTGAGGGTGGAGG | |
| | ATAGGCACGAAGYCAGGTTACGTTGGGTTTGGATCTGAT | |
| | GGAGATGGTGGATTGTGGACCCCACTCCACCCCAAAGC | |
| | CAATCTCTTTTTCTGTTTTTCTTCTTTCGCAGACTCCCAT | |
| | ATGGACCTGGGTTTTACAGATGGGCATTGGCCCAACAAC | |
| | TATCTACTATATCCACTTCCCCTATTTGCTTATCCACTCC | |
| | CCTTTTCAATAAAACAT | |
| S05933 | CCAAAAGCATTATTAAAATTTCAATATCCATACCTTTCC | 78 |
| | AAGCCTTGCAAGGTTCCCAGAMACAGCATCTAATGGAA | |
| | CACTTCCATCAGTCCATTTCTTCTCATGAATGGTGATACC | |
| | CATT | |
| S13078 | TGTCCTTCCCACTCTTGAGTTAAATTCTTGTGATCTTTAC | 82 |
| | ACAAAAACCTGAGCAAGAAACATTAGCCCAAAATGAAT | |
| | CCAACAAGGATGCACCCACCAGACTTGAAAATGATACA | |
| | AGGTTTGGAAAGAATTTGGTTTACACAAGAAGATCAAA | |
| | GGCCATTTCTGAATCAACAGACGCCCAAGAGGCCAATC | |
| | CAACWCCGSTAGATCAGGTAACTAACAATGATTTTCCA | |
| | ATTTTGAATGATAATCTTTTAGCTTCTCCTAATGAAACG | |
| | GAAATTTCAGAACATATTGATGACCTTTATCTTCCCATTT | |
| | CCTTTAGAAAAGGAACCAGAACATGTGCCAAAAAGCCT | |
| | CTCTATCCTCTCTCAAATCTCATTTCATAAATTTTTTCCA | |
| | ACCCATAAAACCTT | |
| S13073 | CCAAATGAAATCTTTGCAAATTTTCTCAATGTCTTTACA | 86 |
| | CACCAAGACTGGCAACTTGGACGCTTGAAGCACCTATG | |
| | CAGGAATATTAAACAAGCAAGCTTGTGCCAAAGTTACC | |
| | CTGCCAGTCGTGGATAGGATCTTAGCTTTCCAGCTAGTA | |
| | ATTTTTCGCCAGATATTTCTAATATGGGGTTTAAACAGC | |
| | TACTCATWTCTCTTATGATCCTATACWAGGAAACCCAA | |
| | ATAGGAATCAAGATTATTAGTAATAGTCACCTGAAGCTG | |
| | TTGACTAAGCTCCCTCACCTTAGCTTATGAGACATTAGT | |
| | TRAGAAGAGAACCTTTGATTTGCTCATGTTGATCTTCTG | |
| | GCTAGATGCATTAGCAAAAGGGTTTAATATCTGGTTGAC | |
| | TTCCCCAGCTGCTA | |
| S01261 | tGCGGGCTAATGTTGTGCTGTGTGTGAGGGTGGGGTCGA | 91 |
| | TGAGATTTAGATTCnnnnnnnTAAGCTACTGTCAAGAATA | |
| | CRTAAAACGTTTAGATCTTAAAACACAAGAAAGACCAGC | |
| | ACTCCAGCATGCAGTCACCACTGTACTCAATGTTTAGAT | |
| | CCTCCWTTTTTCCCGTGGCCATGTCCACCTCTTCCTCCAC | |
| | CACCACCCTTTCCTCTATGATGTTTTTTTGCTCCACCTAA | |
| | TGCCCTGTTAGCATCAGGTCTCACTTGCATCTCTTGTGGC | |
| | AAGGGAAGAATATGATCCACACATTTACGGAAGCTAAA | |
| | ATCATCGACAGATTCATCGTCCCTGACGAGGAAGAAGC | |
| | ACCTACTTTTCCACATAGGGTGCTTGCGGACTTGAAAAA | |
| | CACGGATTCCTCCTCCAATCTTAGTATCAGGTTCTGTGT | |
| | GACCGTTCTTAAGCAACTCCAGTAGCATCATATGTTCAT | |
| | ACTGCCACACAAAAAACAACTCATAGTGAATTCAGCAA | |
| | GTAGATTAAGGTTGTGGGCATCATCAAGACACGTTACTA | |
| | AGACTAAGCCAATTTAAACATTAACTGGCCATGAATTTG | |
| | CAYGCAGTTGAATCTTGAATTGCACATGCATTAAAATAG | |
| | ATTACAAACCATTTTATATCACATGTTGATGGCACTGAA | |
| | AGCCATTTCACAAGCTCATGCATTAAAATAATTGTTTCC | |
| | TAATAATCCTATGCACTTCTGGAAATCTTTGCAGTGAAT | |
| | CTGCCAAACATGATAAATAAATAGATTGTRAAAAAGAG | |
| | AGTTTGCGTACATATCCAACCAAGCTATTCACTAAATTT | |
| | AGCCGGRAAAAATATTTCAAAAA | |
| S14531 | AAAAGTAGGTGCTTCTTCCTCGTCAGGGACGATGAATCT | 96 |
| | GTCGATGATTTTAGCTTCCGTAAATGTGTGGATCATATT | |
| | CTTCCCTTGCCACAAGAGATGCAAGTGAGACCTGATGCT | |
| | AACAGGGCATTAGGTGGAGCAAAAAAACATCATAGAGG | |
| | AAAGGGTGGTGGTGGAGGAAGAGGTGGACATGGCCACG | |
| | GGAAAAAWGGAGGATCTAAACATTGAGTACAGTGGTG | |
| | ACTGCATGCTGGAGTGCTGGTCTTTCTTGTGTTTTAAGAT | |
| | CTAAACGTTTTACGTATTCTTGACAGTAGCTTATTTCATG | |
| | AATCTAAATCTCATCGACCCCACCCTCACACACAGCACA | |
| | ACATTAGCCCGCATGACCTGCAAAGTTTCTTTATGTAAT | |
| | TATTTTGAGTTTT | |
| S01282 | AGGAATWAGATAATTATGACTGCCAGGCAAGTATCAGG | 101 |
| | CACCCTAGGAGCACAaGTCGTCACCTTTACATAAACTAT | |
| | TAACTAACATTTCAACTAAAACTACTCGCCGTTTATTAA | |
| | ATCCATAACAACACGTTTGAGGCTTCCATTGTTTTTCTCA | |
| | AGCAGTCTCTTATTCATCTCTTTGTCACGAAAACCCTGC | |
| | ACACACACCCAATCACTAATCAAAAGAGAGAATCCART | |
| | TGAACAAATTGCTTAGTAAGAGGCGGGATTAGAAGGTA | |
| | AAGAACTTCAAATTGCAGGCATTTTGATAATAAAATAA | |
| | AAGGTGAACAACCAGAACAACTTACCATCTCCTGTAACT | |
| | CTTCAAGCATATGGTCCCACTCAGAAACACCACAGACAT | |
| | CAACACCACAGAGAGCGTCTAGGGATTGGTCCAGATCG | |
| | TACTCATTCTTCCTCAAGATCTCCTTGTTCAAATCAACCT | |
| | GTTTAAAACCCATCTCCTCAAGCTCTTTAAGTAGATTCT | |
| | CCTCCACAGAATTAATTCCTCCACCAGTACCCATAGATG | |
| | ACGATGGCACATCCACGGTTGGGGATTGCTGATTAGATG | |
| | GAACAGCTGGGGTTGTTTCAGACAGATCAATCATGGTCA | |
| | TAGCYKKTT | |
| S14582 | TCTATGAGTGTGATTAACTTGGCCTTCCATTCAAAAATT | 106 |
| | TCCTTTTTCTTTTTCTTTTATAGATATGGTTTAAGTTTTG | |
| | TTCTTTTAAATGTGCAATAGGTGGCATCCAGGAAAAGATC | |
| | AATGGCACCCTTACGTGAGCYTTATAGCTTKGTTACTGT | |
| | CAAGGATTTTGCTAAAGCCTTTCAGATTTCCAGTGACTT | |
| | TTGYACWGTCAAAGGATCCTCAAATTACCAATAACCCC | |
| | TTGTAGAAGTTGCTATCTGGATCTGGAAATGATTCTTTT | |
| | ATTACGATTGTTTAAAAAAATTGTATTGCTACTTCTCTTG | |
| | CGGATGATGCATAAATTATTGGGTTTATGTACTTGTATT | |
| | TCTGTTTATTGTGTACTCGTTCCTTTTATGTGCTCTCTAA | |
| | AATCATAT | |
| S10245 | GGTCAGACTCAACAGTTATTGGTTGACTGACCATATCCA | 110 |
| | AGAAAATGATGTTCACTAACCTTAAAGTGAAGCCATCAT | |
| | TCATAAAATAGCTTATTAGTCATTTCATCACTATAGTAA | |
| | CCCTGCACTAAATCATTCTCATTACCAGCACTTCAAATT | |
| | ACTCATTTCCATAATTCCGTTGATCCACTCCCTTTCCTGT | |
| | TCCTRCCATGGCCCTGATTGGTTGGTGGTGCTCTTCTTTC | |
| | TGCTTTCCTTCAGGTTGCGGTTGACAGGCTTGCTTCTCGT | |
| | CAAGTTCTTGACTTCTTTTGTGCAAGAAAACTCGATGAG | |
| | ATGCTGCTCGGCAAGTTGAACATGAAGCTGCTGTCCATT | |
| | GATGCTCTGGCTGATGATGCAGATCAAAAGCAGTTCAG | |
| | AGATCCACG | |
| S14581 | CTCCGCCGCCGTCGGCGTCGCCCTCTCCTACCTCTCCTTC | 115 |
| | GGCGTCTCCTCCAACCTCCACTTCCTCGTCCCCATGTTCC | |
| | TCGGCTACGCCTCCATGCTCCTCTTTCGTCCCCGATGCG | |
| | GCATCCTCACCTTCTTCCTCGGATTCGGCTACCTCATTGG | |
| | CTGGTTCGTACAATCGAGATCTATTTTGTCATAATCTCAT | |
| | CWCAATTCAATTCTACTTCTCTTTGACCTCGCTCCGCGTT | |
| | TGCGTTTCTCTCTTCTGCAGCCACGTGTATTACATGAGC | |
| | GGTGACGCATGGAAGGAAGGTGGCATCGATGCCACTGG | |
| | TAAGCGCCAATTAATTATTTATTTATTTTTTGAATTGAGG | |
| | AATGAGGGAATTGACTGATTAGTAGTATTTCACTGGTAA | |
| | CGTTAT | |
| S10446 | GGGTTATTTTTCAGTTGACGTCGGCTAGGTTTTTTTTGGT | 120 |
| | CAAGATTAGCCAATGATGTTTTTTTGGCTGACATCGACC | |
| | GATCATGTTTTTTGCCGACATTGTCCAGGGCTATTTTYG | |
| | GCTGATATCGGCTAGGATATTTTCTGGTCAATGTTAGCT | |
| | AGTGATGCTTTTTGGTTGACATCAACTAAAACTATTTTTC | |
| | AAGYGACGTTGATCAGAGCTATTTTTTTCCGATGTCGGC | |
| | TTGGGTCATTGGCACCAACAAAAAATAGCCTCGATCAA | |
| | AGTTAGCCAAAAAAATCCTAGTCGATGTCGGCCAAAAA | |
| | AATAGTCATGGCCGATGTTGGCCAAAAAACATCATCGCT | |
| | TGACGTCGGCCAAAAAGACCCTTGCTGGCATCGGTTAA | |
| | AATAGTCTCGGT | |
| S14561 | AAAAAATAAATTARAAAAAAAAAYCAATTTTACTTGTA | 125 |
| | AGTTTTAATTAGATCGACTACTATTATGTTTTGGTTCCRA | |
| | TAATCAGTTTCAGTTCACTTTTTTTTACAGCAAATTTCGG | |
| | TTCACTTTATGGTTATCTAACCTGATATTATTCGATTCCG | |
| | GTATCGGTTTATAAGTTTGCAAACCCGAATAATCTGATC | |
| | CAAWCCAAACGCAWATACACTGAACTTTCGTTCTAGAG | |
| | CTCTGCAGCACTTCAGCACAATCGTTTTTATTTTTTCTCA | |
| | GGGTACCAGAGCTTCTGCCATCATGGATGGACACCGTGT | |
| | TACTCCAAAAATACAAGATTGATTTGATGATTGAAAAAT | |
| | AAGAATTTAAACATAAAGGAAAAAAGAGAAGTCTCAAG | |
| | ATTAAAATTC | |
| S14552 | ACACGTACATAAGTTATTAAATTTAATTTAAGAGAAAAT | 130 |
| | AATTCATAATATTTAATTATTTTTCATTTAAAGATTGATT | |
| | GAAATCATCAACAAGTGATCATCCRTGTAATAATTTTTT | |
| | GCATATTGAATWTAGTGACTCAAAAAGTTTCGATAAAK | |
| | ATAGGATTAGTTTCATTWGGGTGCTTATATAAAGATCAT | |
| | GAAACSTGAAGCAATTCTGTTGTGTGTGGCTGGACGATC | |
| | ATGGTCCGGACGACCGGGGCAGACGAGGGAAATTGTAC | |
| | TCAGTTAGTTGGTTAATTTCTGTTATAACTTCCATAACAG | |
| | AAATAATTATTTTCTGTTAGAGTCTATGGGTATAAATAC | |
| | ATTGTATTTATCAACTCTTGTACGTTCATGATTGATTAAT | |
| | GCATAAGTCC | |
| S14562 | TGATTAAGCCCAAGTACAACATAAAAAAAAAATACAAA | 135 |
| | ATAACAAATGTATTGGATTGCGCTCGCCCCCCAGTGATC | |
| | TTATCTCTGGTGATTTCGGCCTCCTATTAGCTTCTTAACC | |
| | ATGGTTGTAATCCTAATCACTCTTCTCCCTATGAATTTCT | |
| | TTGCCATGCAAGTGCAATAAAAACCTTCCCAATTTGGGT | |
| | CTCTRAGTTCTAATCCTTTCCAAGATCAGTCAAACCTAA | |
| | AATGAAACAAGACAGTTACACCATAACAAGTCCTAGAA | |
| | TGGAACACGTCAACGGAAAAACACAATGAAATTACACT | |
| | AAAAAGAGAAGAAAAAGCATGGAAACCTAACTAGACA | |
| | ATATAGAGAGATGACATGAGATGCAACGATTAAACAAC | |
| | TAGATCGGTGATGCC | |
| S13012 | ATGTTTGATTCCCAATTCAACCGGTGTAATCGGTCGATC | 140 |
| | GGAGCTAGCTAGAGTCTGATAACATGGTAGTTATAACTT | |
| | ACATCTATTTATTTTTTAGAAAAAAATAAACTAGTAAGT | |
| | TATAACTACAATTGTCTTAAAATATACTTAAGCTGTGGC | |
| | TTACTAACATTAGGGGTGAGCATGGCTCAATCCGGCTTG | |
| | CAAACYTGGATCCACTCGTAGTTGATTTGGATAGGTTTA | |
| | AATTTTTAAATTAGACGATGTATTTTTTAGGATGAGTTT | |
| | GAGTTAAGTTTTGAGTAATCTCAAACTAGTTTTTTTGTCT | |
| | TAATAAGTAGAGCTTGATAAAAAAAAAAAAAAATATAT | |
| | ATATATATATATATATATATATATATATATATATATATTT | |
| | ATCAAAGATT | |
| S05107 | GTCACCCCCACATAAATCAAACTTTACAATATTCTAGTC | 145 |
| | AATGGCCGACATCCACCACATYCAACATAAATAGGTTTT | |
| | GRCGTTGCTTTTATTCTATCACATGCATTGTTCAAAGCTA | |
| | AGA | |

The SNP markers identified in these studies could be useful, for example, for detecting and/or selecting soybean plants with improved tolerance to iron deficiency. The physical position of each SNP is provided in Tables 5 and 8 based upon the JGI Glymal assembly (Schmutz et al. (2010) Nature 463:178-183). Any marker capable of detecting a polymorphism at one of these physical positions, or a marker associated, linked, or closely linked thereto, could also be useful, for example, for detecting and/or selecting soybean plants with improved iron deficiency tolerance. In some examples, the SNP allele present in the tolerant parental line could be used as a favorable allele to detect or select plants with improved tolerance. In other examples, the SNP allele present in the susceptible parent line could be used as an unfavorable allele to detect or select plants without improved tolerance.

These SNP markers could also be used to determine a favorable or unfavorable haplotype. In certain examples, a favorable haplotype would include any combinations of two or more of allele “G” for marker S00405-1-A, allele “T” for marker S15121-001-Q1, allele “A” for marker S15124-001-Q1, and allele “G” for marker S04776-1-A. In addition to the markers listed in Table 2, other closely linked markers could also be useful for detecting and/or selecting soybean plants with improved iron deficiency tolerance. Further, chromosome intervals containing the markers provided herein could also be used, the chromosome interval on linkage group A1 flanked by and including S15081-001 (8712346 bp, 27.94 cM) and S01282-1-A (2012649 bp, 13.45 cM), or an interval flanked by and including BARC-044481-08709 (9097270 bp, 22.52 cM) and BARC-019031-03052 (7546740 bp, 14.63 cM), or an interval flanked by and including the top of LG A1 (0 cM) and Sat 137, 995905 bp, 3.63 cM). In additional examples, the one or more marker locus detected comprises one or more markers within the chromosome interval on linkage group A1 a region of 5 cM, 10 cM, 15 cM, 20 cM, 25 cM, or 30 cM comprising S00405. In still further examples, the one or more marker locus detected comprises one or more markers within the chromosome interval on chromosome 5 (Gm05) flanked by and including nucleotide positions 7677721 and 9097315. Other useful intervals include, for example the interval flanked by and including markers S00405-1 and S01282-1-A on LG-A1, or any interval provided in FIG. 1 or the Tables provided herein.

What is claimed is:

1. A method of detecting a first soybean plant or germplasm with improved iron deficiency tolerance, the method comprising detecting at least one favorable allele of one or more marker locus within 10 cM of a polynucleotide selected from the group consisting of:
a) one or more marker loci on linkage group A1 selected from the group consisting of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107;
b) one or more markers on linkage group A1 selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, S14581-001-Q001, S10446-001-Q1, S14561-001-Q001, S14552-001-Q001, S14562-001-Q001, S13012-001-Q002, and S05107-001-Q002;
c) one or more markers within a genomic DNA region selected from the group consisting of SEQ ID NOs: 1-145;
d) one or more markers within a region selected from the group consisting of SEQ ID NOs: 5, 10, 15, 20, 25, 29, 33, 37, 42, 47, 52, 56, 61, 66, 70, 74, 78, 82, 86, 91, 96, 101, 106, 110, 115, 120, 125, 130, 135, 140, and 145;
e) one or more markers within a chromosome interval on linkage group A1 flanked by and including S15081 and S01282;
f) one or more markers within a chromosome interval on linkage group A1 flanked by and including BARC-044481-08709 and BARC-019031-03052;
g) one or more markers within a chromosome interval on linkage group A1 flanked by and including the top of LG A1 and Sat)137;
h) one or more markers within a chromosome interval on chromosome 5 flanked by and including nucleotide positions 7677721 and 9097315; and,
i) one or more markers within a chromosome interval on linkage group A1 of 30 cM comprising S00405, S15121, or S15124.

2. The method of claim 1, wherein said detecting comprises detection of a haplotype comprising two or more markers selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, S14581-001-Q001, S10446-001-Q1, S14561-001-Q001, S14552-001-Q001, S14562-001-Q001, S13012-001-Q002, and S05107-001-Q002.

3. The method of claim 1, wherein said detecting comprises detection of a haplotype comprising three or more markers selected from the group consisting of S00405-1-A, S15121-001-Q001, S15124-001-Q001, S04776-1-A, S15081-001-Q001, S05017-1-K1, S07022-1-K001, S10456-1-K1, S15126-001-Q001, S15071-001-Q001, S15122-001-Q001, S13062-1-K1, S15125-001-Q001, S15123-001-Q001, S12985-1-K1, S13064-1-K1, S05933-1-K1, S13078-1-K1, S13073-1-K1, S01261-1-A, S14531-001-Q001, S01282-1-A, S14582-001-Q001, S10245-1-K1, S14581-001-Q001, S10446-001-Q1, S14561-001-Q001, S14552-001-Q001, S14562-001-Q001, S13012-001-Q002, and S05107-001-Q002.

4. The method of claim 1, wherein said detecting comprises detection of a haplotype comprising markers S00405-1-A, S15121-001-Q001, S15124-1-Q001, and S04776-1-A.

5. The method of claim 1, wherein said detecting comprises detection of a haplotype comprising markers S00405-1-A and S15124-1-Q001.

6. The method of claim 1, wherein said at least one favorable allele of one or more marker loci is selected from the group consisting of S00405-1-A allele G, Gm05 position 8810680 allele G, S15121-001-Q001 allele T, Gm05 position 8650576 allele T, S15124-001-Q001 allele A, Gm05 position 8671038 allele A, S04776-1-A allele G, Gm05 position 8021614 allele G, S15081-001-Q001 null allele, Gm05 position 8712346 null allele, S05017-1-K1 allele A, Gm05 position 9097414 allele A, S07022-1-K001 allele T, Gm05 position 9002798 allele T, S10456-1-K1 allele A, Gm05 position 8796827 allele A, S15126-001-Q001 allele A, Gm05 position 8809479 allele A, S15071-001-Q001 allele A, Gm05 position 8796827 allele A, S15122-001-Q001 allele G, Gm05 position 8659968 allele G, S13062-1-K1 allele C, Gm05 position 8622812 allele C, S15125-001-Q001 allele T, Gm05 position 8673968 allele T, S15123-001-Q001 allele A, Gm05 position 8660316 allele A, S12985-1-K1 allele A, Gm05 position 8659986 allele A, S13064-1-K1 allele T, Gm05 position 8173288 allele T, S05933-1-K1 allele A, Gm05 position 7943632 allele A, S13078-1-K1 allele G, Gm05 position 7850805 allele G, S13073-1-K1 allele T, Gm05 position 7677721 allele T, S01261-1-A allele A, Gm05 position 620718 allele A, S14531-001-Q001 allele T, Gm05 position 620718 allele A, S01282-1-A allele G, Gm05 position 2012649 allele G, S14582-001-Q001 allele C, Gm05 position 2578312 allele C, S10245-1-K1 allele G, Gm05 position 2573680 allele G, S14581-001-Q001 allele T, Gm05 position 2703606 allele T, S10446-001-Q1 allele A, Gm05 position 3271804 allele A, S14561-001-Q001 allele T, Gm05 position 3603395 allele T, S14552-001-Q001 allele G, Gm05 position 3604317 allele G, S14562-001-Q001 allele G, Gm05 position 3597393 allele G, S13012-001-Q002 allele T, Gm05 position S711938 allele T, S05107-001-Q002 allele T, and Gm05 position 6852084 allele T.

7. The method of claim 5, wherein said haplotype comprises the marker alleles S00405-1-A allele G, S15121-001-Q001 allele T, S15124-1-Q001 allele A, and S04776-1-A allele G.

8. The method of claim 1, wherein the detecting comprises amplifying the marker locus or a portion of the marker locus and detecting the resulting amplified marker amplicon.

9. The method of claim 8, wherein the amplifying comprises:
a) admixing an amplification primer or amplification primer pair with a nucleic acid isolated from the first soybean plant or germplasm, wherein the primer or primer pair is complementary or partially complementary to at least a portion of the marker locus and is capable of initiating DNA polymerization by a DNA polymerase using the soybean nucleic acid as a template; and
b) extending the primer or primer pair in a DNA polymerization reaction comprising a DNA polymerase and a template nucleic acid to generate at least one amplicon.

10. The method of claim 9, wherein the admixing of step 1) further comprises admixing at least one nucleic acid probe.

11. The method of claim 9, wherein the detection comprises PCR analysis.

12. The method of claim 1, further comprising selecting the first soybean plant or germplasm, or selecting a progeny of the first soybean plant or germplasm.

13. The method of claim 12, further comprising crossing the selected first soybean plant or germplasm with a second soybean plant or germplasm.

14. The method of claim 13, wherein the second soybean plant or germplasm comprises an exotic soybean strain or an elite soybean strain.

15. A kit for selecting at least one soybean plant, the kit comprising:
a) primers or probes for detecting one or more marker loci associated with one or more quantitative trait loci associated with improved iron deficiency tolerance, wherein the one or more marker loci are selected from the group consisting of:
i) one or more loci on linkage group A1 selected from the group consisting of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107;
ii) one or more markers within a genomic DNA region selected from the group consisting of SEQ ID NOs: 5, 10, 15, 20, 25, 29, 33, 37, 42, 47, 52, 56, 61, 66, 70, 74, 78, 82, 86, 91, 96, 101, 106, 110, 115, 120, 125, 130, 135, 140, and 145;
iii) one or more markers within the chromosome interval on linkage group A1 flanked by and including S15081 and S01282
iv) one or more markers within the chromosome interval on linkage group A1 flanked by and including BARC-44481-08709 and BARC-019031-03052;
v) one or more markers within the chromosome interval on linkage group A1 flanked by and including the top of LG A1 and Sa_{—}137;
vi) one or more markers within the chromosome interval on chromosome 5 flanked by and including nucleotide positions 7677721 and 9097315; and
vii) one or more markers within a chromosome interval on linkage group A1 of 30 cM comprising S00405, S15121, or S15124; and,
b) instructions for using the primers or probes for detecting the one or more marker loci and correlating the detected marker loci with predicted improved tolerance or increased susceptibility to iron deficiency.

16. The kit of claim 15, wherein the primers or probes comprise one or more of SEQ ID NOs: 1-145.

17. The method of claim 15, wherein the marker locus comprises S00405-1-A and wherein the primers or probes comprise SEQ ID NOs: 1-4.

18. The method of claim 15, wherein the marker locus comprises S15121-001-Q1 and wherein the primers or probes comprise SEQ ID NOs: 6-9.

19. The method of claim 15, wherein the marker locus comprises S15124-001-Q1 and wherein the primers or probes comprise SEQ ID NOs: 11-14.

20. The method of claim 15, wherein the marker locus comprises S04776-1-A and wherein the primers or probes comprise SEQ ID NOs: 16-19.

21. An isolated polynucleotide capable of detecting a marker locus selected from the group consisting of S00405, S15121, S15124, S04776, S15081, S05017, S07022, S10456, S15126, S15071, S15122, S13062, S15125, S15123, S12985, S13064, S05933, S13078, S13073, S01261, S14531, S01282, S14582, S10245, S14581, S10446, S14561, S14552, S14562, S13012, and S05107.

22. The isolated polynucleotide of claim 21, wherein the polynucleotide comprises a nucleotide sequence selected from the group consisting of SEQ ID NOs: 1-145.

23. An isolated polynucleotide capable of detecting a nucleotide polymorphism on soybean chromosome 5 wherein the polymorphism is at a genomic location selected from the group consisting of Gm05 position 8810680, Gm05 position 8650576, Gm05 position 8671038, Gm05 position 8021614, Gm05 position 8712346, Gm05 position 9097414, Gm05 position 9002798, Gm05 position 8796827, Gm05 position 8809479, Gm05 position 8659968, Gm05 position 8622812, Gm05 position 8673968, Gm05 position 8660316, Gm05 position 8659986, Gm05 position 8173288, Gm05 position 7943632, Gm05 position 7850805, Gm05 position 7677721, Gm05 position 620718, Gm05 position 2012649, Gm05 position 2578312, Gm05 position 2573680, Gm05 position 2703606, Gm05 position 3271804, Gm05 position 3603395, Gm05 position 3604317, Gm05 position 3597393, Gm05 position 5711938, and Gm05 position 6852084.

24. A method of soybean plant breeding comprising:
a) crossing at least two different soybean parent plants, wherein the parent soybean plants differ in iron deficiency tolerance;
b) obtaining a population of progeny soybean seed from said cross;
c) genotyping the progeny soybean seed with at least one genetic marker; and,
d) selecting a subpopulation comprising at least one soybean seed possessing a genotype for improved iron deficiency tolerance,
wherein the mean iron deficiency tolerance of the selected subpopulation is improved as compared to the mean iron deficiency tolerance of the non-selected progeny subpopulation.

25. A method of soybean plant breeding comprising:
a) crossing two different soybean parent plants, wherein the parent soybean plants differ in iron deficiency tolerance, and the parent soybean plant with higher iron deficiency tolerance has an earlier maturity adapted for a northern growing region;
b) obtaining progeny soybean seed from said cross;
c) genotyping the progeny seed of said cross with a genetic marker; and,
d) selecting progeny soybean seed possessing a genotype for improved iron deficiency tolerance.

26. The method of claim 25, wherein the parent plants differ in maturity by at least 10 days.

27. The method of claim 26, wherein the selected progeny soybean seed are adapted for a northern growing region.
